(19) United States
(12) Patent Application Publication
(10) Pub. No.: US 2013/0336986 A1
(43) Pub. Date: Dec. 19, 2013

(54) KINASES AS TARGETS FOR ANTI-DIABETIC THERAPY

(71) Applicants: Mathias Falcenberg, Martinsried (DE); Axel Ullrich, Munich (DE)

(72) Inventors: Mathias Falcenberg, Martinsried (DE); Axel Ullrich, Munich (DE)

(73) Assignee: MAX-PLANCK-GESELLSCHAFT ZUR FÖRDERUNG DER WISSENSCHAFTEN E.V., Munich (DE)

(21) Appl. No.: 13/820,226

(22) PCT Filed: Sep. 5, 2011
(86) PCT No.: PCT/EP2011/004567

§ 371 Date: Mar. 1, 2013

Related U.S. Application Data
(60) Provisional application No. 61/344,668, filed on Sep. 8, 2010.

(30) Foreign Application Priority Data
Sep. 3, 2010 (EP) 10075383.9

(51) Int. Cl. A61K 31/713

(52) U.S. Cl. CPC A61K 31/713
USPC 424/158.1; 536/24.5; 536/23.1; 530/300; 530/389.8; 514/44 A; 514/44 R; 514/6.9; 436/501; 435/6.18

(57) ABSTRACT

The present invention is related to compound capable of modulating the activity and/or expression of the protein kinases SCYL1, ADCK1, and GRK5, thereby enhancing the expression and/or release of insulin. The invention is further related to methods of identifying said compounds for the treatment of diseases of the carbohydrate metabolism. The invention is further related to methods of treatment of diseases of the carbohydrate metabolism, particularly diabetes mellitus type 2.

The present invention is related to compound capable of modulating the activity and/or expression of certain protein kinases thereby enhancing the expression and/or release of insulin. The invention is further related to methods of identifying said compounds for the treatment of metabolic diseases. The invention is further related to methods of treatment of metabolic diseases, particularly diabetes mellitus type 2.

Diabetes as a leading cause of death in developed countries is a metabolic condition characterized by high blood sugar levels. There are two main types of diabetes: type 1, resulting from insufficient insulin production of the pancreas beta cells, which requires the person to inject insulin; and type 2, resulting from insensitivity of peripheral tissues (such skeletal muscle, liver or adipose tissue) insulin release alterations, and relative insulin deficiency. Diabetes mellitus type 2 is often acquired and accompanied by obesity; it can be treated in first hand by reducing weight, diet and exercise. Type 1 diabetes is a genetic or autoimmune disease; the only effective therapy to date is the supply of exogenous insulin. This therapy does not cure diabetes; the person needs continuous supply of insulin.

The decreased insulin sensitivity of peripheral tissues in type 2 diabetes which accounts for 90% of all cases of the disease is initially compensated by an increased release of insulin by the beta cells of the pancreas. At a certain stage of the disease, the pancreas cannot maintain the increases release of insulin anymore. As disease progresses, drugs which are currently available and elevate insulin release have lead to beta-cell damage and loss of insulin production.

A number of diseases, including cancer, diabetes and inflammation are linked to perturbation of protein kinase mediated cell signaling pathways. For some time, a new class of multiple kinase drugs has been undergoing clinical trials. Some have been approved for various applications, mostly for the treatment of cancer. The targets of these multiple kinase inhibitors like Imanitib or Sunitinib interact at all stages of signal transduction: from the receptor tyrosine kinases which initiate intracellular signaling to second-messenger generators and kinases involved in signaling cascades and finally to those kinases which regulate the cell cycle governing cellular fate.

Several publications have shown the effect of kinase-inhibitors like Sunitinib (Sutent®) and Imatinib (Gleevec®) on diabetes during a period of treatment which leads to a remission of diabetes type 1 or 2 in patients. However, only few kinases could be identified that affect the insulin release or sensitivity specifically to develop a more specific treatment strategy.

The objective of the present invention is to provide targets for the treatment of metabolic diseases such as diabetes, compounds which are useful for raising the blood insulin level by enhancing the insulin release of the pancreatic beta cells and methods for identifying such compounds. This goal is achieved by the compounds which bind to regulating protein kinases according to claim 1 as well as by the methods for identifying such compounds and the disclosed targets SCYL1, ADCK1, and GRK5. Further advantageous embodiments, aspects and details of the invention are evident from the pending claims, the description, the examples and the figures.

### SHORT DESCRIPTION

The invention refers particularly to a modulator for the inhibition of the activity of protein kinases, wherein kinases with a molar mass larger than 60 kDa selected from the group consisting of SCYL1, ADCK1, and GRK5 are preferred targets for inhibition, and wherein a preferred goal of the inhibition is the treatment of metabolic diseases, more preferred of a disease of the carbohydrate metabolism, more preferred of diabetes, more preferred of diabetes mellitus type 2, and most preferred for the up-regulation of insulin production and/or release of insulin. The invention refers further to a modulator for the inactivation, degradation, downregulation, intercalation of at least one nucleic acid selected from the group consisting of the nucleic acid encoding SCYL1, the nucleic acid encoding ADCK1, and the nucleic acid encoding GRK5 for the treatment of metabolic diseases, more preferred of a disease of the carbohydrate metabolism, more preferred of diabetes, more preferred of diabetes mellitus type 2, and most preferred for the up-regulation of insulin production and/or release of insulin.

The said modulator can be chosen from the group comprising a small molecule, an RNA molecule, a siRNA molecule, a miRNA molecule, or a precursor thereof, an antisense oligonucleotide, an aptamer, a polypeptide, an antibody, or a ribozyme, wherein RNA, peptides, small molecules and aptamers are preferred modulators.

The invention refers further to a pharmaceutical composition comprising a modulator for the treatment of metabolic diseases, more preferred of a disease of the carbohydrate metabolism, more preferred of diabetes, more preferred of diabetes mellitus type 2, and most preferred for the up-regulation of insulin production.

The invention refers further to a method for screening for a modulator for treatment of a metabolic disease, wherein the method comprises providing a test compound for contacting at least one polypeptide or nucleic acid coding for at least one polypeptide of a mass larger than 60 kDa selected from the group consisting of SCYL1, ADCK1, and GRK5 polypeptide, detecting the binding of said test compound to the SCYL1, ADCK1, or GRK5 polypeptide or nucleic acid coding for at least one polypeptide, and determining the activity of the SCYL1, ADCK1, or GRK5 polypeptide in the presence of said test compound.

For identification of an inventive compound the invention further provides a kit comprising the SCYL1, ADCK1, and/or GRK5 polypeptides, the nucleic acid encoding SCYL1, the nucleic acid encoding ADCK1, and the nucleic acid encoding GRK5, a cell line with a glucose dependent insulin production, and a control compound known to affect the insulin production by binding the SCYL1, ADCK1, and/or GRK5 polypeptide.

The invention further provides a method for treatment of a metabolic disease comprising administering a subject in need thereof a therapeutically effective amount of at least one modulator for inhibition or activation of at least one of the kinases selected from the group consisting of SCYL1, ADCK1, and GRK5, or inactivation, degradation, downregulation, intercalation or activation of at least one nucleic acid selected from the group consisting of the nucleic acid encoding SCYL1, the nucleic acid encoding ADCK1, and the nucleic acid encoding GRK5.

### DESCRIPTION

It was surprisingly found that Sunitinib has an effect on insulin release in a dose dependent manner. It was found that this effect was due to the inhibition of certain kinases by Sunitinib. It was further surprisingly found that the inhibition of identified protein kinases of a molar mass larger than 60 kDa by other compounds results in the enhanced release of insulin. It was further found that the combined inhibition of certain protein kinase pairs results in the release of additional insulin. It was further found that the inhibition of certain protein kinase pairs results in an insulin releasing effect which equals the effect after Sunitinib treatment.

Kinases are enzymes which catalyze the transfer of a phosphate group from a donor onto an acceptor. Phosphorylated is a nucleophil functional group, such as hydroxyl-, carboxy-, guanidino-, thiol-, or imidazole groups. Kinases which phosphorylate proteins are called protein kinases. Protein kinases play a particular role in cellular signal transduction. They are usually categorized by their substrate; thus protein kinases may be roughly divided into two groups: protein tyrosine kinases (PTK), which phosphorylate the hydroxyl group of the tyrosine, and serine/threonine kinases (STK), which phosphorylate the hydroxyl groups of the serine or threonine. Examples for PTK are Kinases of the EphA family, Lck, Scyl, HCK, BLK, ITK, TEC, EXK, BTK, CTK, Fyn, Fgr, Src, Yes, Lyn, Tyk, JAK-family, CSK, Arg, Abl, Fes, Fer, Srm, Brk, Syk, ZAP70, FAK, PYK2, DDR, TRK, HER-family, FGFR-family, FLT, Mer, Reg, Axl, Met, Ron, RYK, InsR, IGF1R, LTK, ALK, Ros, Lmr-family. STK are mainly regulated by cAMP, cGMP, DAG, Ca²⁺ or Calmodulin, 1,2-Diacylglycerine, PIP3 and other phospholipid-derivates. Examples for STK include enzymes of the families protein kinase A, B and C, GRK-family, MAST-family, CSNK, PRK, NDR, p70S6K, MSK, MRCK, ROCK, CRIK, DMPK, PKN, Nek, Pim, Aur, SSTK, TSSK, Obscn, skMLCK, DRAK, FAPK, BRSK, MNK, PKD, MAP, PIK3, CHBK, PIP, CERK, TLK, CASK, AKT, KCNH2, GSK, FUK.

Other categorizations are based on the activating compounds of the kinases, or the activating mechanism, or certain catalytic domains or specific amino acid sequences of the kinases. The sum of all kinases in one cell is called kinome.

Based on their function the protein kinases present a very important control mechanism in signal transduction, and are controlling various anabolic and metabolic pathways. On the basis of their importance dysfunctions of protein kinases in cellular pathways are the cause for many diseases, like cancer, metabolic diseases, cardiovascular diseases, arteriosclerosis, thyroid disorders, endocrinological diseases, gastroenterological diseases, inflammation, immune disorders, disorders affecting growth and development, hematological diseases, respiratory diseases, muscle skeleton diseases, neurological diseases, and urological disease. This makes these enzymes attractive molecular targets for therapy.

Surprisingly the kinases SCYL1, ADCK1, and GRK5 were identified to modulate the insulin release in a significant manner (FIGS. 1 and 4). These kinases are preferred targets for the therapy of metabolic disease, preferred of diabetes, more preferred of diabetes type 2, and most preferred to elevate the blood level of insulin. These kinases share the feature of having a molecular mass of at least 60 kDa. Apparently kinases of a certain size are particularly easy inhibited resulting in modulation of insulin release. Kinases which present a target for the therapy of metabolic diseases are basically all kinases which affect metabolic pathways. According to the invention, kinases which have been found to affect or modulate the insulin release or sensitivity and are thus preferred targets for a therapy of diabetes, preferably diabetes type 2, are kinases the inhibition of which is correlated to a significant increase of insulin release, namely SCYL1, ADCK1, and GRK5.

The present invention refers particularly to a modulator for
a) inhibition of at least one of the protein kinases selected from the group consisting of SCYL1, ADCK1, and GRK5, or
b) inactivation, degradation, downregulation, intercalation of at least one nucleic acid selected from the group consisting of the nucleic acid encoding SCYL1, the nucleic acid encoding ADCK1, and the nucleic acid encoding GRK5,
for the treatment of disease of the carbohydrate metabolism.

Thus, in a preferred embodiment of the invention, the action of the protein kinases SCYL1, ADCK1, and/or GRK5 is blocked by a modulator or even more preferred by an inhibitor.

Hence, the present invention refers preferred to an inhibitor for
a) at least one of the protein kinases selected from the group consisting of SCYL1, ADCK1, and GRK5, or
b) inactivation, degradation, downregulation or intercalation of at least one nucleic acid selected from the group consisting of the nucleic acid encoding SCYL1, the nucleic acid encoding ADCK1, and the nucleic acid encoding GRK5,
for the treatment of disease of the carbohydrate metabolism.

It is sufficient to block one of the kinases SCYL1, ADCK1, or GRK5. However, inhibition of two kinases of SCYL1, ADCK1, GRK5 may further increase the therapeutic effect. Consequently, in a further preferred embodiment of the invention, two of the kinases are inhibited in combination for an increased insulin release. The possible combinations are SCYL1/ADCK1, SCYL1/GRK5, and ADCK1/GRK5. In a further preferred embodiment of the invention, all three enzymes SCYL1, ADCK1, and GRK5 may be inhibited at once for an increased insulin release. In order to achieve the inhibition of two of the kinases a single modulator or inhibitor or a combination of two modulators or inhibitors could be used. In order to achieve the inhibition of the three kinases SCYL1, ADCK1, and GRK5 a single modulator or inhibitor or a combination of two modulators or inhibitors or a combination of three inhibitors could be used. Enzyme inhibitors are, in general, molecules which bind to enzymes and decrease their activity. The binding of an inhibitor can stop a substrate from entering the enzymes active site, compete with the substrate for the binding site, or hinder the enzyme from catalyzing its reaction. Inhibitor binding can be reversible or irreversible. Protein kinase inhibitors are a type of enzyme inhibitors which specifically block the action of one or more protein kinases. Inhibition of protein kinases can be achieved using a pseudosubstrate binding to the active site of these kinases mimicking the target sequence of the corresponding kinase, but having no serine or threonine.

In another preferred embodiment, the action of the protein kinases SCYL1, ADCK1, and/or GRK5 is impeded by interference of their nucleic acid, which can be both DNA and RNA, by inactivation, degradation, downregulation, or intercalation. Inactivation of a nucleic acid can happen for instance by methylation of nucleotides, insertion, deletion, nucleotide exchange, cross linkage, or strand break/damage. Nucleic acids can be degraded down to single nucleotides by temperature, chemicals, enzymes, and particularly RNA by deadenylation or 5′ decay or 3′ decay. Downregulation of DNA or RNA is referred to as diminished expression of these nucleic acids and can happen by binding of repressors, which are usually polypeptides, but can also happen by chemical or structural changes or modifications of the nucleic acids. Intercalation is the reversible inclusion of a molecule between two other molecules. In nucleic acids, intercalation occurs when ligands of an appropriate size and chemical nature fit themselves in between base pairs.

The term modulator as it appears herein refers to a molecule that is able to change the activity of the SCYL1, ADCK1, and/or GRK5 polypeptides. This change may be an increase or a decrease in enzymatic activity, binding characteristics, or functional, immunological or any other biological property of the polypeptides. In order to enhance the insulin release, a decrease of the enzymatic activity is advantageous.

According to the invention, modulators for the inhibition of SCYL1, ADCK1, and GRK5 can be molecules like small molecules, RNA or DNA molecules, siRNA or precursor thereof, miRNA or precursors thereof, ribozymes, DNA or RNA antisense oligonucleotides, aptamers, antibodies or fragments thereof, peptides, polypeptides, cyclopeptides, or drugs like imatinib, dasanitib, and sorafenib.

The inventive modulators are also referred to as compounds or test compounds. They modulate the expression and/or activity of the polypeptides of the invention and can be identified using one or more assays, alone or in combination. Test compounds used in the screening are not particularly limited. They can be either artificial or natural.

The term small molecule refers to low molecular weight organic compound which is by definition not a polymer. In the field of pharmacology, it is usually restricted to a molecule that also binds with high affinity to a biopolymer such as proteins, nucleic acids, or polysaccharides. The upper molecular weight limit for a small molecule is approximately 200 Da which allows for the possibility to rapidly diffuse across cell membranes. Small molecules are broadly used as enzyme inhibitors, thus they are preferred modulators for the inhibition of the preferred kinases in the present invention.

Small interfering RNA (short interfering RNA, silencing RNA, siRNA) is a class of double-stranded RNA-molecules, which are 19-30 nucleotides, preferably 20-25 nucleotides long. SiRNAs are involved in the RNA-interference of the expression of a specific gene. SiRNAs are cut from long doublestranded RNAs by the RNase III Dicer. They can also be derived by chemical synthesis. They also play a role in antiviral mechanisms or in shaping the chromatin structure of a genome. In molecular research, synthetic siRNAs can also be used in RNA-interference (RNAi) to regulate down the expression of specific target genes. With their ability to knock down essentially any gene of interest, siRNAs have been used to knock down protein kinases to investigate their role in insulin production (FIG. 1-FIG. 4). SiRNAs are preferred modulators for inhibition of the preferred kinases in the present invention.

MicroRNAs (miRNAs) are posttranscriptional regulators that bind to complementary sequences in the 3′UTR of mRNA transcripts, usually resulting in gene silencing. They are short RNA molecules which are about 22 nucleotides long. As miRNAs have been shown to play multiple roles in transcript degradation, sequestering and transcriptional suppression, they are also preferred modulators for inhibition of the preferred kinases in the present invention.

Precursor molecules, e.g. precursor molecules of siRNA and/or miRNA may be a substrate for the siRNA/miRNA-biogenesis-apparatus of the target cell. This comprises, for example, RNA precursor molecules such as double-stranded RNA (dsRNA) or short hairpin RNA-molecules (shRNA), which are processed by endonucleases such as Drosha and/or Pasha to siRNA-molecules or miRNA-molecules, respectively. For this reason, for example dsRNA-molecules or short hairpin RNA-molecules (shRNA) having a length of more than 27 nucleotides, preferably more than 30 up to 100 nucleotides or longer, and mostly preferred dsRNA-molecules having a length of 30-50 nucleotides, can be used.

Further precursor molecules according to the invention may be DNA constructs encoding dsRNA, shRNA, siRNA and/or miRNA, whereby the coding elements are controlled by regulatory elements allowing an expression of dsRNA, shRNA, siRNA and/or miRNA in the target cell. Examples for such control elements are polymerase II promoters or polymerase III promoters such as, for example, U6 or H1.

Ribozymes are catalytic RNAs which possess a well defined structure that enables them to catalyze a chemical reaction. Apart from naturally occurring ribozymes they can be made artificially and be tailored to interact with nucleic acids and proteins. Ribozymes are also preferred modulators for inhibition of the preferred kinases in the present invention.

Antisense oligonucleotides are single strands of DNA or RNA that are complementary to a chosen sequence. They are between 10 and 35 nucleotides long, preferably about 20-25 nucleotides. Antisense DNA oligonucleotides can target specific, complementary RNA, and upon binding DNA/RNA hybrids are formed. Antisense RNA oligonucleotides can bind to mRNA by binding to mRNA strands. Antisense oligonucleotides are also preferred modulators for inhibition of the preferred kinases in the present invention.

Aptamers are oligonucleic acid (DNA or RNA aptamers) or peptide molecules (peptide aptamers) that bind to a specific target molecule. Aptamers can be used for therapeutic purposes as macromolecular drugs. Aptamers can be created by selecting them from a large random sequence pool. Aptamers are also preferred modulators for inhibition of the preferred kinases in the present invention.

Antibodies are proteins which bind very specifically to antigens. They are formed by the immune system of the body in response to antigen presence. They can be formed for virtually any structure and are thus valuable tools for direct interaction with certain molecules. Recombinant techniques are used to generate antibodies and antibody fragments which basically consist of the binding moieties of the antibodies, such as single chain antibodies. They can be applied in vivo in extracellular and intracellular applications. Antibodies are also preferred modulators for inhibition of the preferred kinases in the present invention. Various antibodies binding to the kinases SCYL1, ADCK1 and GRK5 are commercially available. Alternatively, specific inhibitor antibodies against the kinases can by generated by technology known in the art, so that antibody generation does not represent an undue experimental burden for use of the invention.

Peptides are stretches of amino acid residues which are connected by peptide bonds. They can be seen as little proteins. Peptides are usually up to 100 amino acids long, from which on the compound is referred to as a protein. Polypeptides are peptides of at least 10 amino acids. Cyclopeptides are formed by two, three or more amino acids, which form ring structures and have thus no C- and N-terminal amino acids. Peptides are preferred, polypeptides more preferred modulators for inhibition of the preferred kinases in the present invention.

The drugs sunitinib, imatinib, dasatinib, and sorafenib are small molecules which inhibit protein kinases which are mainly used in cancer treatment. However, in the present invention the drug sunitinib is not a preferred modulator for inhibition of the preferred kinases as one major side effect under sunitinib treatment is high blood pressure. People who have diabetes tend to have more trouble with high blood pressure than people who don't have the disease. Having both diabetes and high blood pressure can pack a damaging one-two punch as far as increasing the risk of heart disease, stroke, and eye, kidney and nerve complications. There are particularly common diabetes complications associated with elevated blood pressure. These complications include diabetic retinopathy and diabetic nephropathy. Controlling blood pressure of people with diabetes reduces the risk of future complications as established by a study done by the UK Prospective Diabetes Study.

Metabolic diseases refer to diseases and conditions characterized by pathological disorders of the metabolism. They are mainly characterized by enzyme defects and abnormalities in the regulating system leading to a pathological enrichment of substrates, lack of metabolic products, failure of producing energy, of regeneration of cellular constituents, of elimination of metabolic products, and of maintenance of homeostasis. They can be acquired or be a genetic disease. Metabolic disorders include, but are not limited to, obesity and diabetes (e.g., diabetes type I, diabetes type II, MODY, and gestational diabetes), hypoglycemia, amyloidosis, branched chain disease, hyperaminoacidemia, hyperaminoaciduria, disturbances of the metabolism of urea, hyperammonemia, mucopolysaccharidoses e.g. Maroteaux-Lamy syndrom, glycogen storage diseases and lipid storage diseases, Cori's disease, intestinal carbohydrate malabsorption, maltase-, lactase-, sucrase-insufficiency, disorders of the metabolism of fructose, disorders of the metabolism of galactose, galactosaemia, disturbances of pyruvate metabolism, hypolipidemia, hypolipoproteinemia, hyperlipidemia, hyperlipoproteinemia, camitine or camitine acyltransferase deficiency, porphyrias, disturbances of the purine metabolism, lysosomal diseases, metabolic diseases of nerves and nervous systems like gangliosidoses, sphingolipidoses, sulfatidoses, leucodystrophies, Lesch-Nyhan syndrome, dysfunction of the parathyroid glands, pancreatic islet cell dysfunction, carbohydrate and lipid storage myopathies, glycogenoses, myoglobinuria, alkaptonuria, adrenogenital syndrome, ketosis, ketoacidosis, methylmalonaciduria, Morbus Addison, Morbus Conn, Morbus Cushing, Morbus Fabry, Morbus Gaucher, Morbus Hunter, cystic fibrosis, phenylketonuria, thesaurismosis, uricopathia. Carbohydrate metabolism denotes the various biochemical processes responsible for the formation, breakdown and interconversion of carbohydrates in living organisms, wherein the most important carbohydrate is glucose. The hormone insulin is the primary regulatory signal in animals; when present, it causes many tissue cells to take up glucose from the circulation, causes some cells to store glucose internally in the form of glycogen, causes some cells to take in and hold lipids, and in many cases controls cellular electrolyte balances and amino acid uptake as well. Diseases of the carbohydrate metabolism refer to diseases and conditions characterized in pathophysiological alterations in the metabolism of one or more carbohydrates. It is preferred if the disease of the carbohydrate metabolism is chosen of one disease of the group comprising or consisting of Diabetes mellitus, Lactose intolerance, Fructose intolerance, Galactosemia, Glycogen storage disease, diabetic ketoacidosis, hyperosmolar coma and hypoglycemia.

The invention relates also to pharmaceutical compositions comprising or consisting of an effective amount of at least one inventive compound, and at least one pharmaceutically acceptable carrier, excipient, binders, disintegrates, glidents, diluents, lubricants, coloring agents, sweetening agents, flavoring agents, preservatives, solvent or the like. The pharmaceutical compositions of the present invention can be prepared in a conventional solid or liquid carrier or diluents and a conventional pharmaceutically-made adjuvant at suitable dosage level in a known way.

According to the invention, the inventive compound or the pharmaceutical composition can be used for the treatment of diseases of the carbohydrate metabolism, preferably of diabetes mellitus, more preferably of diabetes mellitus type 2, and most preferably to increase the level of insulin release from pancreas cells.

The inventive pharmaceutical composition is formulated to be compatible with its intended route of administration. Administration forms include, for example, pills, tablets, film tablets, coated tablets, capsules, liposomal formulations, micro- and nano-formulations, powders and deposits. Furthermore, the present invention also includes pharmaceutical preparations for parenteral application, including dermal, intradermal, intragastral, intracutan, intravasal, intravenous, intramuscular, intraperitoneal, intranasal, intravaginal, intrabuccal, percutan, rectal, subcutaneous, sublingual, topical, or transdermal application, which preparations in addition to typical vehicles and/or diluents contain the compound according to the present invention. Intravenous and oral applications are preferred forms of administration in the present invention, wherein oral application is particularly preferred.

The present invention also includes the mammalian milk, artificial mammalian milk as well as mammalian milk substitutes as a formulation for oral administration of the inventive compound to newborns, toddlers, and infants, either as pharmaceutical preparations, and/or as dietary food supplements.

The inventive compound can also be administered in form of its pharmaceutically active salts. Suitable pharmaceutically active salts comprise acid addition salts and alkali or earth alkali salts. For instance, sodium, potassium, lithium, magnesium or calcium salts can be obtained.

The pharmaceutical compositions according to the present invention will typically be administered together with suitable carrier materials selected with respect to the intended form of administration, i.e. for oral administration in the form of tablets, capsules (either solid filled, semi-solid filled or liquid filled), powders for constitution, aerosol preparations consistent with conventional pharmaceutical practices. Other suitable formulations are gels, elixirs, dispersible granules, syrups, suspensions, creams, lotions, solutions, emulsions, suspensions, dispersions, and the like. Suitable dosage forms for sustained release include tablets having layers of varying disintegration rates or controlled release polymeric matrices impregnated with the active components and shaped in tablet form or capsules containing such impregnated or encapsulated porous polymeric matrices. The pharmaceutical compositions may be comprised of 5 to 95% by weight of the inventive compound.

As pharmaceutically acceptable carrier, excipient and/or diluents can be used lactose, starch, sucrose, cellulose, magnesium stearate, dicalcium phosphate, calcium sulfate, talc, mannitol, ethyl alcohol (liquid filled capsules).

Suitable binders include starch, gelatin, natural sugars, corn sweeteners, natural and synthetic gums such as acacia, sodium alginate, carboxymethyl-cellulose, polyethylene glycol and waxes. Among the lubricants that may be mentioned for use in these dosage forms, boric acid, sodium benzoate, sodium acetate, sodium chloride, and the like. Disintegrants include starch, methylcellulose, guar gum and the like. Sweetening and flavoring agents and preservatives may also be included where appropriate. Some of the terms noted above, namely disintegrants, diluents, lubricants, binders and the like, are discussed in more detail below.

Additionally, the compositions or modulators of the present invention may be formulated in sustained release form to provide the rate controlled release of any one or more of the components or active ingredients to optimize the therapeutic effects. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidify.

Also included are solid form preparations which are intended to be converted, shortly before use, to liquid form preparations for either oral or parenteral administration. Such liquid forms include solutions, suspensions and emulsions.

The inventive compound may also be deliverable transdermally. The transdermal compositions may take the form of creams, lotions, aerosols and/or emulsions and can be included in a transdermal patch of the matrix or reservoir type as are conventional in the art for this purpose.

The term capsule refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

Tablet means compressed or molded solid dosage form containing the active ingredients with suitable diluents. The tablet can be prepared by compression of mixtures or granulations obtained by wet granulation, dry granulation or by compaction well known to a person skilled in the art.

Oral gels refer to the active ingredients dispersed or solubilized in a hydrophilic semi-solid matrix.

Powders for constitution refer to powder blends containing the active ingredients and suitable diluents which can be suspended in water or juices. One example for such an oral administration form for newborns, toddlers and/or infants is a human breast milk substitute which is produced from milk powder and milk whey powder, optionally and partially substituted with lactose.

Human breast milk is a complex fluid, rich in nutrients and in non-nutritional bioactive components. It contains all of the nutrients needed by the newborn baby. These include the metabolic components (fat, protein, and carbohydrates), water, and the raw materials for tissue growth and development, such as fatty acids, amino acids, minerals, vitamins, and trace elements.

More than 98% of the fat is in the form of triglycerides. Oleic acid and palmitic acid are the most abundant fatty acids in breastmilk triglycerides, with comparatively high proportions of the essential fatty acids, and linolenic acid, followed by long-chain polyunsaturated fatty acids, such as arachidonic acid and docosahexaenoic acid. These long-chain fatty acids are constituents of brain and neural tissue and are needed in early life for mental and visual development. The lipid component of breast milk is the transport vehicle for fat-soluble micronutrients such as prostaglandins and vitamins A, D, E, and K.

Proteins account for approximately 75% of the nitrogen-containing compounds in breast milk. Non-protein nitrogen substances include urea, nucleotides, peptides, free amino acids, and DNA. The proteins of breast milk can be divided into two categories: micellar caseins and aqueous whey proteins, present in the ratio of about 40:60. Casein forms micelles of relatively small volume and produces a soft, flocculent curd in the infant's stomach. The major whey proteins are lactalbumin, lactoferrin, secretory IgA, and serum albumin, with a large number of other proteins and peptides present in smaller amounts.

The principal carbohydrate is lactose, a disaccharide produced in the mammary epithelial cell from glucose by a reaction involving lactalbumin.

In addition to the nutritional components, breast milk contains a wealth of bioactive components that have beneficial non-nutritional functions. These include a wide range of specific and non-specific antimicrobial factors; cytokines and anti-inflammatory substances; and hormones, growth modulators, and digestive enzymes, many of which have multiple activities. These components may be of particular importance for young infants because of the immaturity of the host defense and digestive systems early in life.

The artificial mother milk formulations or mother milk substitutes of the present invention are preferably prepared by adding to a mother milk formulation including commercially available mother milk formulations especially in powder form of the compound of the present invention. The inventive compound is preferably added in an amount of 3-100 μg compound or per 100 ml (commercially available) mother milk formulation, more preferably in an amount of 5-70 μg/100 ml and most preferably in an amount of 10-40 μg/100 ml mother milk formulation.

Suitable diluents are substances that usually make up the major portion of the composition or dosage form. Suitable diluents include sugars such as lactose, sucrose, mannitol and sorbitol, starches derived from wheat, corn rice and potato, and celluloses such as microcrystalline cellulose. The amount of diluents in the composition can range from about 5 to about 95% by weight of the total composition, preferably from about 25 to about 75%, more preferably from about 30 to about 60% by weight, and most preferably from about 40 to 50% by weight.

The term disintegrants refers to materials added to the composition to help it break apart (disintegrate) and release the medicaments. Suitable disintegrants include starches, “cold water soluble” modified starches such as sodium carboxymethyl starch, natural and synthetic gums such as locust bean, karaya, guar, tragacanth and agar, cellulose derivatives such as methylcellulose and sodium carboxymethylcellulose, microcrystalline celluloses and cross-linked microcrystalline celluloses such as sodium croscarmellose, alginates such as alginic acid and sodium alginate, clays such as bentonites, and effervescent mixtures. The amount of disintegrant in the composition can range from about 1 to about 40% by weight of the composition, preferably 2 to about 30% by weight of the composition, more preferably from about 3 to 20% by weight of the composition, and most preferably from about 5 to about 10% by weight.

Binders characterize substances that bind or “glue” powders together and make them cohesive by forming granules, thus serving as the “adhesive” in the formulation. Binders add cohesive strength already available in the diluents or bulking agent. Suitable binders include sugars such as sucrose, starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropyl-methylcellulose; polyvinylpyrrolidone; and inorganics such as magnesium aluminum silicate. The amount of binder in the composition can range from about 1 to 30% by weight of the composition, preferably from about 2 to about 20% by weight of the composition, more preferably from about 3 to about 10% by weight, even more preferably from about 3 to about 6% by weight.

Lubricant refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d′l-leucine. Lubricants are usually added at the very last step before compression, since they must be present on the surfaces of the granules and in between them and the parts of the tablet press. The amount of lubricant in the composition can range from about 0.05 to about 15% by weight of the composition, preferably 0.2 to about 5% by weight of the composition, more preferably from about 0.3 to about 3%, and most preferably from about 0.3 to about 1.5% by weight of the composition.

Glidents are materials that prevent caking and improve the flow characteristics of granulations, so that flow is smooth and uniform. Suitable glidents include silicon dioxide and talc. The amount of glident in the composition can range from about 0.01 to 10% by weight of the composition, preferably 0.1% to about 7% by weight of the total composition, more preferably from about 0.2 to 5% by weight, and most preferably from about 0.5 to about 2% by weight.

Coloring agents are excipients that provide coloration to the composition or the dosage form. Such excipients can include food grade dyes and food grade dyes adsorbed onto a suitable adsorbent such as clay or aluminum oxide. The amount of the coloring agent can vary from about 0.01 to 10% by weight of the composition, preferably from about 0.05 to 6% by weight, more preferably from about 0.1 to about 4% by weight of the composition, and most preferably from about 0.1 to about 1%.

Liquid form preparations include solutions, suspensions and emulsions. As an example may be mentioned water or water-propylene glycol solutions for parenteral injections or addition of sweeteners and opacifiers for oral solutions, suspensions and emulsions. Liquid form preparations may also include solutions for intranasal administration.

Other preferred pharmaceutical compositions are buffered solutions. The term buffer, buffer system, buffer solution and buffered solution, when used with reference to hydrogen-ion concentration or pH, refers to the ability of a system, particularly an aqueous solution, to resist a change of pH on adding acid or alkali, or on dilution with a solvent. Preferred buffer systems can be selected from the group consisting of formate (pKa=3.75), lactate (pKa=3.86), benzoic acid (pKa=4.2) oxalate (pKa=4.29), fumarate (pKa=4.38), aniline (pKa=4.63), acetate buffer (pKa=4.76), citrate buffer (pKa2=4.76, pKa3=6.4), glutamate buffer (pKa=4.3), phosphate buffer (pKa=7.20), succinate (pKa1=4.93; pKa2=5.62), pyridine (pKa=5.23), phthalate (pKa=5.41); histidine (pKa=6.04), MES (2-(N-morpholino)ethanesulphonic acid; pKa=6.15); maleic acid (pKa=6.26); cacodylate (dimethylarsinate, pKa=6.27), carbonic acid (pKa=6.35), ADA (N-(2-acetamido)imino-diacetic acid (pKa=6.62); PIPES (4-piperazinebis-(ethanesulfonic acid; BIS-TRIS-propane (1,3-bis[tris(hydroxymethyl)methylamino]-propane), pKa=6.80), ethylendiamine (pKa=6.85), ACES 2-[(2-amino-2-oxoethyl)amino]ethanesulphonic acid; pKa=6.9), imidazole (pKa=6.95), MOPS (3-(N-morphin)-propansulfonic acid; pKa=7.20), diethylmalonic acid (pKa=7.2), TES (2-[tris(hydroxymethyl)methyl]amino ethanesulphonic acid; pKa=7.50) and HEPES (N-2-hydroxylethylpiperazin-N′-2-ethansulfonic acid; pKa=7.55) buffers or other buffers having a pKa between 3.8 to 7.7.

Preferred is the group of carboxylic acid buffers such as acetate and carboxylic diacid buffers such as fumarate, tartrate and phthalate and carboxylic triacid buffers such as citrate. Another group of preferred buffers is represented by inorganic buffers such as sulfate, borate, carbonate, oxalate, calcium hydroxyde and phosphate buffers. Another group of preferred buffers are nitrogen containing buffers such as imidazole, diethylenediamine, and piperazine.

Also preferred are sulfonic acid buffers such as TES, HEPES, ACES, PIPES, [(2-hydroxy-1,1-bis(hydroxymethyl)ethyl)amino]-1-propanesulfonic acid (TAPS), 4-(2-hydroxyethyl)piperazine-1-propanesulfonic acid (EPPS), 4-Morpholinepropanesulfonic acid (MOPS) and N,N-bis(2-hydroxyethyl)-2-aminoethanesulfonic acid (BES).

Another group of preferred buffers are glycine buffers such as glycine, glycyl-glycine, glycyl-glycyl-glycine, N,N-bis(2-hydroxyethyl)glycine and N-[2-hydroxy-1,1-bis(hydroxy-methyl)ethyl]glycine (Tricine).

Preferred are also amino acid buffers such as glycine, alanine, valine, leucine, isoleucine, serine, threonine, phenylalanine, tyrosine, tryptophane, lysine, arginine, histidine, aspartate, glutamate, asparagine, glutamine, cysteine, methionine, proline, 4-hydroxyproline, N,N,N-trimethyllysine, 3-methylhistidine, 5-hydroxylysine, O-phosphoserine, γ-carboxyglutamate, ε-N-acetyllysine, ω-N-methylarginine, citrulline, ornithine and derivatives thereof.

Preferred are the buffers having an effective pH range of from 2.7 to 8.5, and more preferred of from 3.8 to 7.7. The effective pH range for each buffer can be defined as pKa−1 to pKa+1, where Ka is the ionization constant for the weak acid in the buffer and pKa=−log K.

Most preferred are buffers suitable for pharmaceutical use e.g. buffers suitable for administration to a patient such as acetate, carbonate, citrate, fumarate, glutamate, lactate, phosphate, phthalate, and succinate buffers. Particularly preferred examples of commonly used pharmaceutical buffers are acetate buffer, citrate buffer, glutamate buffer and phosphate buffer. Also most preferred is the group of carboxylic acid buffers. The term “carboxylic acid buffers” as used herein shall refer to carboxylic mono acid buffers and carboxylic diacid buffers as well as carboxylic triacid buffers. Of course also combinations of buffers, especially of the buffers mentioned herein are useful for the present invention.

Some suitable pharmaceutical buffers are a citrate buffer (preferably at a final formulation concentration of from about 20 to 200 mM, more preferably at a final concentration of from about 30 to 120 mM) or an acetate buffer (preferably at a final formulation concentration of about 20 to 200 mM) or a phosphate buffer (preferably at a final formulation concentration of about 20 to 200 mM).

Techniques for the formulation and administration of the compound of the present invention may be found in “Remington's Pharmaceutical Sciences” Mack Publishing Co., Easton Pa. A suitable composition comprising the compound mentioned herein may be a solution of the compound in a suitable liquid pharmaceutical carrier or any other formulation such as tablets, pills, film tablets, coated tablets, dragees, capsules, powders and deposits, gels, syrups, slurries, suspensions, emulsions, and the like.

A particularly preferred pharmaceutical composition is a lyophilised (freeze-dried) preparation (lyophilisate) suitable for administration by inhalation or for intravenous administration. To prepare the preferred lyophilised preparation the compound of the invention is solubilised in a 4 to 5% (w/v) mannitol solution and the solution is then lyophilised. The mannitol solution can also be prepared in a suitable buffer solution as described above.

Further examples of suitable cryo-/lyoprotectants (otherwise referred to as bulking agents or stabilizers) include thiol-free albumin, immunoglobulins, polyalkyleneoxides (e.g. PEG, polypropylene glycols), trehalose, glucose, sucrose, sorbitol, dextran, maltose, raffinose, stachyose and other saccharides (cf. for instance WO 97/29782), while mannitol is used preferably. These can be used in conventional amounts in conventional lyophilization techniques. Methods of lyophilisation are well known in the art of preparing pharmaceutical formulations.

For administration by inhalation the particle diameter of the lyophilised preparation is preferably between 2 to 5 μm, more preferably between 3 to 4 μm. The lyophilised preparation is particularly suitable for administration using an inhalator, for example the OPTINEB® or VENTA-NEB® inhalator (NEBU-TEC, Elsenfeld, Germany). The lyophilised product can be rehydrated in sterile distilled water or any other suitable liquid for inhalation administration.

Alternatively for intravenous administration the lyophilised product can be rehydrated in sterile distilled water or any other suitable liquid for intravenous administration.

After rehydration for administration in sterile distilled water or another suitable liquid the lyophilised preparation should have the approximate physiological osmolality of the target tissue for the rehydrated compound preparation i.e. blood for intravenous administration or lung tissue for inhalation administration. Thus it is preferred that the rehydrated formulation is substantially isotonic.

The preferred dosage concentration for either intravenous, oral, or inhalation administration is between 100 to 2000 pmol/ml, and more preferably is between 200 to 800 pmol/ml. These are also the preferred ranges of the compound in the mother milk substitute or artificial mother milk formulation or the pharmaceutical compositions disclosed herein.

Still another aspect of the present invention relates to the use of the inventive compound as a dietary supplement. That dietary supplement is preferably for oral administration and especially but not limited to administration to newborns, toddlers, and/or infants. A dietary supplement is intended to supplement the diet. The “dietary ingredients” in these products may in addition include: vitamins, minerals, herbs or other botanicals, amino acids, and substances such as enzymes, organ tissues, glandulars, and metabolites. Dietary supplements may be manufactured in forms such as tablets, capsules, softgels, gelcaps, liquids, or powders.

The invention further relates to a method for screening for a modulator for treatment of a metabolic disease, the method comprising
a) contacting a test compound with at least one polypeptide selected from the group consisting of SCYL1, ADCK1, and GRK5 polypeptide,
b) detecting the binding of said test compound to the SCYL1, ADCK1, or GRK5 polypeptide, and
c) determining the activity of the SCYL1, ADCK1, or GRK5 polypeptide in the presence of said test compound.

The screening method of the present invention apparently consists of three steps. The term test compound may be any of the potential modulators listed above. The contacting of the test compound with at least one of the polypeptides can happen e.g. in the form of a compound library, in physiological or non-physiological solution, or solid phase systems, however a liquid environment is preferred. The conditions and the time need to be sufficient to allow the test compound to bind to the polypeptide(s). The method is normally carried our in solution at room temperature and at a suitable pH value normally between pH 5 and 9, all parameters which are easily selected by a skilled person.

The polypeptides SCYL1, ADCK1, and/or GRK5 can be obtained by purification from primary human cells, cell lines, from cells which have been transfected with expression constructs which contain the nucleic acid sequences encoding one or more of the polypeptides SCYL1, ADCK1, and/or GRK5, or by direct chemical synthesis.

The nucleic acid sequences encoding the polypeptides SCYL1, ADCK1, and/or GRK5 can be obtained by cloning the relevant genes, amplification of the cDNAs or chemical synthesis of the nucleic sequences. For the expression of the corresponding polypeptides the nucleic acid sequences can be inserted into expression vectors, such as recombinant bacteriophage, plasmid, or cosmid DNA expression vectors.

The term binding refers to an interaction between the test compound and one or more of the polypeptides SCYL1, ADCK1, and/or GRK5 or the nucleic acids encoding one or more of the polypeptides SCYL1, ADCK1, and/or GRK5. For binding to a protein, the binding interaction is dependent upon the presence of a particular structure of the kinase, e.g. the antigenic determinant or epitope, recognized by the binding molecule. For binding of compounds to nucleic acids, test compounds need to have a complementary sequence to the nucleic acids, or fit into certain secondary or tertiary structures of the nucleic acids.

The binding of the test compounds to the polypeptides or nucleic acids can be checked by any convenient method known in the art. A separation step may be included to separate bound from unbound components. To check whether the test compound has been bound by the polypeptide or nucleic acid, it is advantageous if the test compound is labeled for direct detection (radioactivity, luminescence, fluorescence, optical or electron density etc.) or indirect detection (e.g., epitope tag such as the FLAG, V5 or myc epitopes, an enzyme tag such as horseradish peroxidase or luciferase, a transcription product, etc.). The label may be bound to a substrate, to the proteins employed in the assays, or to the candidate pharmacological agent. The binding of a test compound can also be conveniently checked if one of the components is immobilized on a solid substrate. The substrate can be made of a wide variety of materials and in various shapes, e.g. tubes, microtiter plates, microbeads, dipsticks and the like. It is also advantageous if one of the components is modified by biotinylation, so that the components can be immobilized on streptavidin-covered surfaces.

Protein-DNA interactions can be for instance checked by gel shift or band shift assays or elektrophoretic mobility shift assays (EMSA), which is based on the observation that complexes of protein and DNA migrate through a non-denaturing polyacrylamide gel more slowly that a free DNA fragments.

Protein-RNA interactions can be investigated by RNA electrophoretic mobility shift assays which are an in vitro technique used to detect protein-RNA interactions through changes in migration speed during gel electrophoresis. After incubation, the binding reaction is then separated via non-denaturing polyacrylamide gel electrophoresis. Like protein-DNA complexes, a protein-RNA complex migrates more slowly than a free RNA probe through a gel matrix. This causes a migration shift relative to the nonbound RNA probe. Specificity is determined through a competition reaction, where excess unlabeled RNA is incubated in the binding reaction, resulting in a decrease in the shifted signal if the labeled and unlabeled RNA sequences compete for binding of the same protein. Alternatively, the protein-RNA complex may be crosslinked and the reaction run on a denaturing gel. Specificity is determined through visualization of a single shifted band. Traditionally, RNA probes are radioactively labeled for detection, although fluorescent and chemiluminescent detection is also possible. Non-radioactive RNA end-labeling techniques are limited, but more versatile biotin and fluorescent labeling methods are now available. Alternatively, RNA Pull-down assays can be carried out which selectively extract a Protein-RNA complex from a sample. This method has the advantage that several RNAs can be used with the target protein(s), and selectively binding RNAs can be identified. Typically, the RNA pull-down assay takes advantage of high affinity tags, such as biotin or azido-phosphine chemistry. RNA probes can be biotinylated, complexed with a protein from a cell lysate and then purified using agarose or magnetic beads. Alternatively, the protein may be labeled, or the RNA-Protein complex may be isolated using an antibody against the protein of interest. The RNA is then detected by Northern blot or through RT-PCR analysis and the proteins detected by Western blotting or mass spectrometry. Protein-RNA interactions can also be identified by oligonucleotide-targeted RNase H protection assays (RPA), which is a powerful method for detecting RNA and RNA fragments in cell extracts. Unlike Northern blotting or RT-PCR analysis, RPA assays allow greater flexibility in the integrity of target RNA, requiring very short segments for hybridization and detection. RPA assays can also be used to map protein-RNA interactions. In this adaptation of the RPA, RNase H is used to cleave a target RNA molecule at a specific site hybridized with a DNA probe. If a protein is bound to the RNA at the target sequence, it will prevent will block probe hybridization, prevent cleavage by RNase H and indicate a site of interaction between protein and RNA. RNase H requires only a four basepair hybrid with a DNA probe in order to cleave the RNA molecule of interest. Using many small probes allows the entire sequence of RNA to be mapped for sites sites of interaction.

The interactions between peptides and proteins, respectively, can be investigated by various methods, which include, but are not limited to, protein binding microarray, antibody microarrays, protein chips, and a variety of assays, UV-crosslink experiments.

The interactions between nucleic acids can be checked for instance by hybridization, which is based on the annealing of complementary DNA-DNA or DNA-RNA or RNA-RNA-sequences. The nucleotide sequences encoding SCYL1, ADCK1, and GRK5 may be labeled by standard methods and added to a sample of nucleic acids to be used as test compounds under conditions suitable for the formation of hybridization complexes. After a suitable incubation period, the sample is washed and the signal is quantified and compared with a standard value. If the amount of signal in the patient sample is significantly altered from that of a comparable control sample, the nucleotide sequences have hybridized with nucleotide sequences in the sample, and the presence of altered levels of nucleotide sequences encoding GRK5 in the sample indicates the presence of the associated disorder. Such assays may also be used to evaluate the efficacy of a particular therapeutic treatment regimen in animal studies, in clinical trials, or in monitoring the treatment of an individual patient. Interactions between nucleic acids can also be investigated by microarrays. A further way of testing the binding between nucleic acids is the use of gel shift assays, in which hybrid molecules are moving slower in a denaturating gels in electrophoresis.

In all methods to identify compounds that modulate (stimulate or inhibit) the expression and kinase activity of the polypeptides of the invention, the expression level and kinase activity are compared to those detected in the absence of the test compound. The present invention is related particularly to the identification of compounds which have inhibitory activity on the kinase activity of the polypeptides of the invention. Consequently, it is particularly the inhibition of expression and activity that is measured.

The inhibition of nucleic acids on the mRNA-level encoding the polypeptides can be checked by investigating the expression of the polypeptides by quantitative methods, e.g. Western blot or enzyme-linked immune-adsorbent assay (ELISA). A way to quantify the protein expression is further the measuring of fusion proteins, wherein the polypeptides of the invention are fused to proteins or protein fragments which are easy to quantify, like fluorescent proteins. The inhibition of DNA and thus the production of mRNA can be checked by mRNA-quantification. Levels of mRNA can be quantitatively measured by Northern blotting. Another way is the reverse transcription quantitative polymerase chain reaction (RT-PCR followed by qPCR). Another way of quantifying mRNA is the use of microarrays, which are, however, more practical if a large set of mRNAs is investigated.

The inhibition of the polypeptides on the protein-level can be investigated by measuring their activity. The determination of the activity of a polypeptide/protein/enzyme depends on its specificity. Consequently, the activity of kinases is measured in phosphorylation assays, wherein a substrate is phosphorylated by a kinase. The kinase activity of SCYL1, ADCK1, and GRK5 can be detected, for example, by adding ATP having radioactively labeled phosphate to the system containing the polypeptides SCYL1, ADCK1, and/or GRK5 and the substrate and measuring the radioactivity of the phosphate attached to the substrate.

According to the invention, the effect of a test compound on the kinase activity of the polypeptides of the invention can be estimated in a system using an insulin-producing cell line, or primary cells, which are or are derived from pancreatic cells. Therein the change of the insulin release level compared to the level without the compound. The release level of insulin can be estimated with the mRNA and protein quantification levels identified above.

Polypeptides of SCYL1, ADCK1, and/or GRK5 can be used in high-throughput screens to assay test compounds for the ability to modulate the kinase activity. These compounds can be further screened against a functional kinase to determine the effect of the compound on the kinase activity. Further, these compounds can be tested in animal or invertebrate systems to determine activity/effectiveness. Compounds can be identified that activate (agonist) or inactivate (antagonist) the kinase to a desired degree. Further, SCYL1, ADCK1, and/or GRK5 can be used to screen a compound for the ability to stimulate or inhibit interaction between the kinase protein and a molecule that normally interacts with the kinase protein, e.g. a substrate or a component of the signal pathway that the kinase protein normally interacts (for example, another kinase). Such assays typically include the steps of combining the kinase protein with a candidate compound under conditions that allow the kinase protein, or fragment, to interact with the target molecule, and to detect the formation of a complex between the protein and the target or to detect the biochemical consequence of the interaction with the kinase protein and the target, such as any of the associated effects of signal transduction such as protein phosphorylation, cAMP turnover, and adenylate cyclase activation, etc.

Polypeptides of SCYL1, ADCK1, and/or GRK5 are also useful in competition binding assays in methods designed to discover compounds that interact with the kinase (e.g. binding partners and/or ligands). Thus, a compound is exposed to a kinase polypeptide under conditions that allow the compound to bind or to otherwise interact with the polypeptide. Soluble kinase polypeptide is also added to the mixture. If the test compound interacts with the soluble kinase polypeptide, it decreases the amount of complex formed or activity from the kinase target. This type of assay is particularly useful in cases in which compounds are sought that interact with specific regions of the kinase.

To facilitate the identification of modulators of the expression and activity of the peptides of the invention, the invention further provides, in a preferred embodiment, a kit comprising
a) SCYL1, ADCK1, and/or GRK5 polypeptides,
and/or
b) a nucleic acid encoding SCYL1, a nucleic acid encoding ADCK1, and/or a nucleic acid encoding GRK5,
and
c) a control compound known to affect the insulin production by binding the SCYL1, ADCK1, and/or GRK5 polypeptide or the corresponding nucleic acid.

In a further preferred embodiment, the invention provides a kit comprising
a) the SCYL1, ADCK1, and/or GRK5 polypeptides,
and/or
b) the nucleic acid encoding SCYL1, the nucleic acid encoding ADCK1, and/or the nucleic acid encoding GRK5
and
c) a control compound known to affect the insulin production by binding the SCYL1, ADCK1, and/or GRK5 polypeptide or the corresponding nucleic acid, and further comprising
d) a cell line with insulin production.

In a further preferred embodiment, the invention provides a kit comprising
a) the SCYL1, ADCK1, and GRK5 polypeptides,
and/or
b) the nucleic acid encoding SCYL1, the nucleic acid encoding ADCK1, and the nucleic acid encoding GRK5
and
c) a control compound for each of the kinases SCYL1, ADCK1, and GRK5 known to affect the insulin production by binding the SCYL1, ADCK1, and GRK5 polypeptide or the corresponding nucleic acid.

In a further preferred embodiment, the invention provides a kit comprising
a) SCYL1, ADCK1, and GRK5 polypeptides,
and/or
b) a nucleic acid encoding SCYL1, a nucleic acid encoding ADCK1, and a nucleic acid encoding GRK5,
and
c) a control compound for each of the kinases SCYL1, ADCK1, and GRK5 known to affect the insulin production by binding the SCYL1, ADCK1, and GRK5 polypeptide or the corresponding nucleic acid,
and further comprising
d) a cell line with insulin production.

In all embodiments of the kit, the control compounds can be any of the test compounds characterized above. A control compound is used as a reference for the binding/inhibitory efficiency of a test compound because it is known for its binding to a chosen polypeptide or the corresponding nucleic acid which encode the chosen polypeptide, thereby inhibiting the activity or the expression of the polypeptides. A chosen control compound refers to the same polypeptide for which inhibitory compounds are tested; e.g. if compounds for the inhibition of SCYL1 are tested, then the control compound is one which inhibits SCYL1.

In a further preferred embodiment, it is particularly preferred if the kit comprises the polypeptides SCYL1, ADCK1, GRK5 and/or their corresponding nucleic acids.

Control compounds that affect the insulin release by binding to the polypeptides of SCYL1, ADCK1, and GRK5 are e.g. Sunitinib and inventive siRNAs or any other compound which has proved to modulate the insulin production.

The invention is further related to a method for treatment of a disease of the carbohydrate metabolism, preferably diabetes mellitus, more preferably diabetes mellitus type 2, and most preferably for increasing the level of insulin release from pancreas cells comprising:
administering a subject in need thereof a therapeutically effective amount of at least one modulator for:
a) inhibition or activation of at least one of the tyrosine kinases selected from the group consisting of SCYL1, ADCK1, and GRK5 or
b) inactivation, degradation, downregulation, intercalation or activation of at least one nucleic acid selected from the group consisting of the nucleic acid encoding SCYL1, the nucleic acid encoding ADCK1, and the nucleic acid encoding GRK5.

An inventive compound known to affect the expression and/or activity of the polypeptides of SCYL1, ADCK1, and GRK5 can be used for the treatment of a metabolic disease, preferably diabetes mellitus, more preferably diabetes mellitus type 2, and most preferably for increasing the level of insulin release from pancreas cells by administration of the inventive compound(s) within pharmaceutical compositions as outline above. In a preferred embodiment of the invention, at least two of SCYL1, ADCK1, and GRK5 are modulated by one or more of the inventive compounds.

The inventive compounds or inventive compositions are according to the invention useful for each single disease of the group of diseases consisting of metabolic diseases, preferably diseases of the carbohydrate metabolism.

The influence of the kinases SCYL1, ADCK1, and GRK5 on insulin release suggests a particular, but not limited to, utilization of the polypeptides for diagnosis of disease of the carbohydrate metabolism, preferably of diabetes mellitus, more preferably of diabetes mellitus type 2.

The embodiments in the description and the following examples are provided by way of illustration of the invention and are not included for the purpose of limiting the invention. The variations and changes of the invention which are obvious to a person skilled in the field and solutions equivalent to embodiments described herein fall within the scope of protection of the patent claims.

TABLE 1
Sequence identities of the target genes and target proteins

| SeqIdNo | Sequence Name | Gene Accession |
|---|---|---|
| 8 | SCYL1 gene | NM_023912 |
| 9 | GRK5 gene | NM_018869 |
| 10 | ADCK1 gene | NM_028105 |
| 11 | SCYL1 protein | NM_023912 |
| 12 | GRK5 protein | NM_018869 |
| 13 | ADCK1 protein | NM_028105 |

### EXAMPLES

### Example 1

### siRNA Screen in the Pancreatic Beta Cell Line Beta-TC6

To identify the kinases, which might be responsible for the elevated insulin release after Sunitinib treatment, we performed a kinome wide siRNA knock-down screen. The effect on the insulin release of each kinase depletion was monitored by using a rat/mouse insulin ELISA. The resulting data were compared to Sunitinib treatment (5 μM) as positive control and correlated to a non-targeting siRNA. Candidate genes were limited by using hierarchical clustering and by proposing a significant in-/decrease of the insulin release by 15%. Depletion of SCY1-like 1 (SCYL1), aarF-containing kinase 1 (ADCK1), and G protein-coupled receptor kinase 5 (GRK5), resulted in an increase of the insulin release in beta-TC6 cells compared to the control siRNA, rendering those kinases as potential negative modulators of insulin release (FIG. 1).

### Example 2

### Validation of the Negative Modulators

For the validation of the negative modulating kinases SCYL1, GRK5, and ADCK1, the target genes were depleted using four different siRNA-sequences each (FIG. 2A-D). The gene-depletion was measured via mRNA-levels after 72 hours while the insulin release was measured using a rat/mouse insulin ELISA after two hours incubation. The depletion of the residual kinases SCYL1, GRK5, and ADCK1 led to an increased insulin release with different efficiencies. Furthermore, the insulin release for the kinases inversely correlated with the respective knock-down efficiency of SCYL1 and ADCK1 which ranged from 50 to nearly 100% as estimated by RT-PCR and scanning densitometry. In case of GRK5 where all sequences lead to equal knock-down efficiency, this correlation could not be observed. The increase was highest for the depletion of SCYL1 (46.38±5.51%), followed by GRK5 (41.23±1.53%), and ADCK1 (33.95±9.02%). Sunitinib was included as a control (FIG. 3). This enhances the role of those kinases in triggering the insulin release.

TABLE 2
Sequences of the primers used in RT-PCR for target validation

| SeqIdNo | GeneSymbol | Primer | GeneAccession | Sequence 5′ - 3′ |
|---|---|---|---|---|
| 1 | SCYL1 | Fwd | NM_023912 | CGGCGGCGACGATGTGGTTCTTT |
| 2 | SCYL1 | Rev | NM_023912 | CGGCGTTGCCCTGTGCCGAGTA |
| 3 | ADCK1 | Fwd | NM_028105 | CTGACACGGGCAAGGCTGAGATT |
| 4 | ADCK1 | Rev | NM_028105 | GCGCCCTGATACAACACCGAGAC |
| 5 | GRK5 | Fwd | NM_018869 | GCCGGGTGCTGGAGACTGAGGA |
| 6 | GRK5 | Rev | NM_018869 | TGGCGGTTCTGGAGGCTGACTTCT |

### Example 3

### Results Double Knock-Down of the Validated Kinase

Compared to the Sunitinib treatment, the effect of the single knock-downs was less pronounced suggesting the involvement of multiple kinases in triggering the insulin release. The candidate kinases are widespread in various signaling pathways. To investigate whether the kinases have a redundant or additive effect on insulin release, we performed double-knock-downs for each possible kinase pair. The insulin increase due to the double knock-down was correlated to the single knock-downs as well as to the non-targeting siRNA. Out of 16 kinase pairs, SCYL1 and ADCK1 depletion resulted in the highest insulin release (90.64±17.32%), which was equal to the Sunitinib induced insulin release (FIG. 4). For the other kinase pairs, no increased insulin release compared to the single-knock-downs was observed (data not shown).

### Example 4

### Phosphorylation of AKT1 Upon Reduction of Kinase C Candidate Gene Expression in Beta-TC6 Cells

The gene expression of SCYL1, GRK5, and ADCK1 was inhibited by siRNA. Downregulation of SCYL1, GRK5 as well as ADCK1 increased phosphorylation of AKT with a tolerable standard deviation (SCYL1: 43.73±3.37%; GRK5: 96.53±28.87%; ADCK1:) (FIG. 5). It can be concluded that the siRNA mediated reduction of the gene expression results in increased AKT1 phosphorylation. As already mentioned, this increase seems to be connected to insulin release according to the publication by Leibiger B. and colleagues (Leibiger et al. FASEB J, 2010, 24; 1824-1837).

### Example 5

### Measurement of SCYL1, GRK5, and ADCK1 mRNA Levels Upon 24 h Sunitinib Treatment

Beta TC6 cells were treated with 1 μM and 5 μM, respectively, Sunitinib for 24 h. The inhibition of gene expression was estimated by measurement of mRNA levels. Sunitinib treatment negatively influences mRNA levels of candidate kinases (FIG. 6). This observation might represent a further explanation for the positive effect of Sunitinib on diabetes patients in clinics where Sunitinib is applied for a longer period of time.

### Example 6

### Uptake of the Fluorescent Glucose Analogue 2-NBDG Upon Candidate Kinase Knock-Down in Beta C2C12 and 3T3-L1 Cells

The glucose uptake of cells was investigated in response to reduction of candidate gene expression. Down-regulation of GRK5 remarkably affects uptake of 2-NBDG in C2C12 (GRK5: 21.24±3.96%) whereas ADCK1 showed an increase of 9.72±3.81%. In 3T3-L1 cells the reduction of gene expression for SCYL1 gene reduction shows an impact on glucose uptake (9.45±3.45%) (FIG. 7). All data are presented as mean values (±SEM). It can be concluded that the reduction of the G protein-coupled receptor kinase 5 (GRK5) and ADCK1 expression seems to enhance uptake of the glucose analogue 2-NBDG (21.24±3.96%) in mouse myoblast cells (C2C12) without need of insulin stimulation, thus GRK5 seems to trigger the insulin sensitivity and/or insulin independent glucose uptake.

### Example 7

### Phosphorylation of AKT1 Upon Candidate Target Knock-Down in C2C12 and 3T3-L1 Cells

The phosphorylation of AKT1 was investigated upon the reduction of gene expression of SCYL1, GRK5, and ADCK1. As illustrated, downregulation of SCYL1 as well as GRK5 increases phosphorylation of AKT1 in C2C12 cells. In the 3T3-L1 model system we detected a decrease of AKT1 phosphorylation for all candidate kinases (FIG. 8). It can be concluded that in peripheral tissues AKT1 phosphorylation is connected to GLUT4 translocation and therefore to glucose uptake. In the beta-TC6 cell line, the knock-down of SCYL1 and GRK5 increases phosphorylation of AKT1. Furthermore, the results of the 2-NBDG uptake assay in C2C12 cells as well as the observed increase in AKT1 phosphorylation in the beta-TC6 upon reduction of candidate gene expression might explain the functional role of GRK5 in regard to an elevated release of insulin.

### Example 8

### Analysis of a Potential Glucose Mediated Effect on Insulin Released by Beta TC6 Cells

The influence of external glucose was investigated at glucose concentrations ranging from 1.125 to 4.5 mM relative to glucose free control (FIG. 9). The addition of different glucose concentrations ranging from physiological to pathophysiological concentrations (ranging from 1 g/L (5.5 mM) up to 4.5 g/L (25.5 mM)) to media of beta-TC6 cells does not results in a concentration dependent increase in insulin release. However, using concentrations under 0.8 g/L (4.5 mM) glucose displayed a glucose dependent release of insulin (Poitout et al., Diabetes, 1995, 4; 306-313). Thus a glucose concentration of 0.2 g/L (1.125 mM) was chosen for further experiments with a low glucose environment.

### Example 9

### Insulin Released by Beta T6 Cells after Reduction of Gene Expression in Low Glucose Media

The insulin release was mediated by reduction of gene expression in a low glucose environment of 0.2 g/L (1.125 mM) (FIG. 10). It can be concluded that knock-down of SCYL1, GRK5, and ADCK1 seems to decrease the insulin release in a low glucose environment. A performed MTT-assay displayed no impaired cell viability.

### Example 10

### GRK5 as Anti-Diabetic Target for Drug Development

8800 Customer compounds were screened for their ability to inhibit the enzymatic activity of GRK5. Screening was performed using the ADP Glo™ Kinase Assay technology (Promega). The small molecular weight compounds were delivered at 10 mM stock concentration and were tested at a final assay concentration of 100 μM. A narrow hit distribution was observed with an average inhibition of 3.2% and a standard deviation of 11%. In consequence compounds with an inhibition value above 36.1% (=inhav+(3×inhstdev)) are recommended to be considered as hit. The top candidate compounds are summarized in table 3. All five compounds identified by the screen have a molecular weight in the range of 200 to 450 g per mol.

TABLE 3
Top candidates revealed by the screen

| Compound | Inhibition of Grk5 [%] |
|---|---|
| C1 | 68.0 |
| C2 | 60.0 |
| C3 | 56.0 |
| C4 | 52.0 |
| C5 | 51.0 |

IC₅₀ values were determined in triplicates at 12 concentrations with a dilution factor of three. Triplicates were measured on three different assay plates. The maximal compound assay concentration was set to 100 μM representing the compound assay concentration used in the primary screen. A larger maximal compound assay concentration could not be achieved since the maximal compound concentration on the compound plates was 10 mM (>1% DMSO concentration in the assay). For all assay plates z prime values were found to be significantly larger than 0.5 proving statistical relevance of the data (see Table 4). Since the majority of all primary screening hits showed less than 50% GRK5 inhibition at 100 μM primary screening concentration, also the majority of all IC₅₀ graphs did not reach 50% inhibition at the maximal compound concentration of 100 μM. For these compounds the IC₅₀ values were extrapolated if the at least 30% GRK5 inhibition was observed at the maximal compound concentration of 100 μM. For 62% of the examined compounds a valid IC₅₀ value could be determined. The remaining 38% of the compounds had less than 30% inhibition at the highest compound concentration of 100 μM (FIG. 11, Table 4).

TABLE 4
IC50 values of the 5 top candidates

| Copound_ID | IC50 [μM] | ZPRIME (assay plate 1/2/3) | Comment |
|---|---|---|---|
| C1 | 72 | 0.87/0.90/0.92 | |
| C2 | 111 | 0.87/0.90/0.92 | * |
| C3 | 111 | 0.87/0.90/0.92 | * |
| C4 | 71 | 0.87/0.90/0.92 | |
| C5 | >100 | 0.87/0.90/0.92 | |
| Sutent | 169 | 0.87/0.90/0.92 | * |

### Example 11

### Release of Insulin after Inhibition of GRK5 by Compounds 1-5 in a Beta TC6-Cell System

Insulin release in beta-TC6 cells was increased by GRK5 inhibitor treatment. The inhibition by compound two (C2), three (C3) and five (C5) led to the most remarkable effect of elevating insulin release by doubling the effect of Sunitinib (SUT) in our system (C2: 2.58±0.94 at 5 μM and 2.01±0.19 at 10 μM; C3: 3.52±1.35 at 5 μM and 2.15±0.59 at 10 μM; C5: 1.93±0.5 at 5 μM and 1.69±0.19 at 10 μM) (FIG. 12). The values are also displayed in table 5.

TABLE 5
Averages and SEM of FIG. 12

| SUT_10 μM | SUT_5 μM | C5_10 μM | C5_5 μM | C4_10 μM | C4_5 μM |
|---|---|---|---|---|---|
| 1.65 | 1.61 | 1.69 | 1.93 | 1.38 | 1.37 |
| 0.14 | 0.31 | 0.19 | 0.50 | 0.18 | 0.14 |

| C3_10 μM | C3_5 μM | C2_10 μM | C2_5 μM | C1_10 μM | C1_5 μM | DMSO |
|---|---|---|---|---|---|---|
| 2.15 | 3.52 | 2.01 | 2.58 | 1.52 | 1.29 | 1 |
| 0.59 | 1.35 | 0.19 | 0.94 | 0.06 | 0.08 | 0 |

It was shown that all of the GRK5-screen based inhibitors stimulate insulin release in the beta TC6 cell system. Thus the target GRK5 has been validated as important regulator of insulin secretion. Furthermore, the inhibition by compounds two, three and five led to the most remarkable effect of elevating insulin release by doubling the effect of Sunitinib in our system (C2: 2.58±0.94 at 5 μM and 2.01±0.19 at 10 μM; C3: 3.52±1.35 at 5 μM and 2.15±0.59 at 10 μM; C5: 1.93±0.5 at 5 μM and 1.69±0.19 at 10 μM).

### Example 12

### Insulin Released after Blocking of Protein Biosynthesis with Cycloheximid and GRK5 Inhibitor Treatment

It was found that the impact of GRK5 inhibitor on insulin release is not based on intensified insulin synthesis. The insulin release was measured upon cycloheximid (CHX) and appropriate 5 μM compound treatment compared to control treated with cycloheximid and DMSO in mean values (±SEM) (FIG. 13). It can be concluded that blocking of the protein transcription and thus of renewing insulin by protein biosynthesis does not affect insulin release upon exposure to the GRK5 inhibitor compounds.

### Example 13

### Phosphorylation of AKT1 in Beta-TC6 Cells by GRK5 Inhibitor

The phosphorylation of AKT1 was measured upon GRK5 inhibitor treatment. The beta-TC6 cells were treated by 5 μM respectively 10 μM of appropriate compound and compared to a DMSO control as well as to AKT protein levels. It can be concluded that AKT1 is phosphorylated by a 1 μg/mL insulin treatment and on that the phosphorylation of Akt1 is elevated by treatment with GRK5 inhibitor (FIG. 14). In contrast to this, Sunitinib—as a control—decrease the insulin mediated increase in AKT1 phosphorylation.

Together with previously described knock down studies for AKT phosphorylation and 2-NBDG uptake we suggest, that GRK5 plays a role in glucose uptake and thus leads to increased insulin release.

### Example 14

### Insulin Released by Beta TC6 Cells after Inhibition of GRK5 in Low Glucose Environment (1.25 mM)

The inhibition of GRK5 in a low glucose environment led to a decreased insulin secretion after a 5 μM treatment (FIG. 15) and 10 μM treatment (FIG. 16). It can be concluded that the inhibition of GRK5 by 5 μM respectively 10 μM of the revealed compounds led to a decrease of insulin release in a low glucose media (1.125 mM). A performed MTT-assay displayed no impaired cell viability.

### Example 15

### Glucose Uptake Via 2-NBDG after GRK5 Compound Treatment in C2C12, TC6, 3T3-L1 and Cells

Inhibition of GRK5 led to an increased uptake of the fluorescent glucose analogue 2-NBDG in C2C12 (FIG. 17) and TC6 (FIG. 18) cells for all five compounds. We suggest that inhibition of GRK5 leads to increased glucose sensitivity without need of insulin. Thus blocking of GRK5 phosphorylation could substitute for insulin.

The increase of glucose uptake in beta TC6 cells incline with previously observed AKT phosphorylation and insulin release. Somehow, the glucose uptake in 3T3-L1 pre-adipocytes (FIG. 18) could only be stimulated by compound 2, suggesting that the cell model is probably not adequate or should be differentiated.

The data are summarized in Table 6:

TABLE 6
Averages and SEM of FIG. 17-19

| | C2C12 | | beta TC6 | | 3T3-L1 | | |
|---|---|---|---|---|---|---|---|
| | Ratio | SE | Ratio | SE | Ratio | SE | |
| DMSO | 1 | 0 | 1 | 0 | 1 | 0 | n = 3 |
| C1 | 2.346 | 0.53 | 1.567 | 0.12 | 0.989 | 0.07 | |
| C2 | 2.127 | 0.44 | 1.529 | 0.07 | 1.111 | 0.03 | |
| C3 | 2.325 | 0.46 | 1.683 | 0.21 | 1.022 | 0.03 | |
| C4 | 1.774 | 0.43 | 1.284 | 0.09 | 0.930 | 0.07 | |
| C5 | 1.47 | 0.17 | 1.259 | 0.02 | 0.926 | 0.04 | |
| Sut | 1.511 | 0.4 | 1.084 | 0.19 | 0.533 | 0.10 | |

### Example 16

### Glucose Uptake Via 2-NBDG after GRK5 Compound Treatment in Matured C2C12 Myotubes and 3T3-L1

Inhibition of GRK5 led to an increased uptake of the fluorescent glucose analogue 2-NBDG in matured C2C12 myotubes (FIG. 20) and 3T3-L1 adipocytes (FIG. 21) for all five compounds (summarized in table 4). In line with C2C12 cells, the matured myotubes display an increase 2-NBDG uptake after inhibition of GRK5 by compounds 1-5. Moreover, also matured adipocytes respond to all five compounds suggesting that glucose metabolism for adipocytes is basically higher and thus could be detected easily.

TABLE 7
Averages and SEM of FIG. 20 and 21

| | C2C12 matured myotubes | | 3T3-L1 matured adipocytes | |
|---|---|---|---|---|
| | Ratio | SE | Ratio | SE |
| DMSO | 1 | 0 | 1 | 0 |
| C1 | 1.976 | 0.101 | 2.520 | 0.071 |
| C2 | 2.383 | 0.637 | 2.234 | 0.085 |
| C3 | 2.457 | 0.567 | 2.141 | 0.031 |
| C4 | 1.829 | 0.396 | 1.583 | 0.043 |
| C5 | 2.033 | 0.210 | 1.491 | 0.043 |

n = 2

### DESCRIPTION OF THE FIGURES

FIG. 1: Assortment of the positive and negative regulating kinases of the kinome screen. The candidate kinases were limited by using hierarchical clustering and proposing and increased insulin release of 15% significant. Additionally, the data were correlated to Sunitinib treatment (5 μM) and a non-targeting siRNA as positive and negative controls. The results are shown for n≧4 biological independent experiments.

FIG. 2: Correlation of the insulin release increase to the target depletion for each used siRNA sequence for the kinases SCYL1 (FIG. 2A), GRK5 (FIG. 2B), and ADCK1 (FIG. 2C). The insulin release for the kinases (bar chart; upper panel) correlated with their knock-down efficiency, which was monitored by RT-PCR and scanning densitometry (agarose gel; middle panel; Δ depletion; lower panel). The insulin increase as well as the knock-down efficiency is compared to the insulin release or gene depletion of the non-targeting siRNA.

FIG. 3: Validation of insulin release increase for kinases SCYL1, GRK5 and ADCK1. SCYL1 showed the most reliable increase in insulin release after gene-depletion with about 46.38±5.51%, followed by GRK5 (41.23±1.53%), and ADCK1 (33.95±9.02%). The control Sunitinib resulted in 76.52±13%.

FIG. 4: Additional increase of the insulin release due to a double knock-down for the kinase pair SCYL1 and ADCK1. The depletion resulted in the highest Insulin release (90.64±17.32%), which equaled the insulin after Sunitinib treatment. The figure depicts the values of the single knock-down (light grey and grey), the theoretical mathematical value (changeover dark grey to grey) and the real insulin increase after a double knock-down (dark grey).

FIG. 5: Phosphorylation of AKT1 upon reduction of gene expression of SCYL1, GRK5 and ADCK1.

FIG. 6: Measurement of SCYL1, GRK5 and ADCK1 mRNA levels upon Sunitinib treatment of 24 h.

FIG. 7: Uptake of the fluorescent glucose analogue 2-NBDG upon candidate kinase gene knock-down in beta C2C12 and 3T3-L1 cells.

FIG. 8: Phosphorylation of AKT1 upon candidate target knock-down in C2C12 and 3T3-L1 cells.

FIG. 9: Analysis of a potential glucose-mediated effect on insulin released by beta TC6 cells (1.125 to 4.5 mM Glucose).

FIG. 10: Insulin released by beta TC6 cells after reduction of gene expression in low glucose media.

FIG. 11: IC₅₀ graphs of GRK5 primary hit compounds.

FIG. 12: Release of Insulin after inhibition of GRK5 by compounds 1-5 in a beta TC6-cell system.

FIG. 13: Insulin released after blocking of protein biosynthesis with cycloheximid and GRK5 inhibitor treatment.

FIG. 14: Phosphorylation of AKT in beta-TC6 cells by GRK5 Inhibitor.

FIG. 15: Insulin released by beta TC6 cells after inhibition of GRK5 in low glucose environment (1.25 mM) with 5 μM compound.

FIG. 16 Insulin released by beta TC6 cells after inhibition of GRK5 in low glucose environment (1.25 mM) with 10 μM compound.

Legend FIG. 17-21:
1=wo 2-NBDG+compound (glucose free medium+compound);
2=wo 2-NBDG (glucose free medium); 3=100 μM 2-NBDG
4=100 μM 2-NBDG+compound
Upper panel left to right shows always compounds 1-3, lower panel left to right compounds 4-5 and Sunitinib. (FIGS. 20 and 21: Sunitinib is missed 9

FIG. 17: Uptake of glucose analogue 2-NBDG after inhibition of GRK5 in C2C12 mouse myoblasts.

FIG. 18: Uptake of glucose analogue 2-NBDG after inhibition of GRK5 in beta TC6 cells.

FIG. 19: Uptake of glucose analogue 2-NBDG after inhibition of GRK5 in beta 3T3-L1 cells.

FIG. 20: Uptake of glucose analogue 2-NBDG after inhibition of GRK5 in matured C2C12 myotubes.

FIG. 21: Uptake of glucose analogue 2-NBDG after inhibition of GRK5 in 3T3-L1 adipocytes.

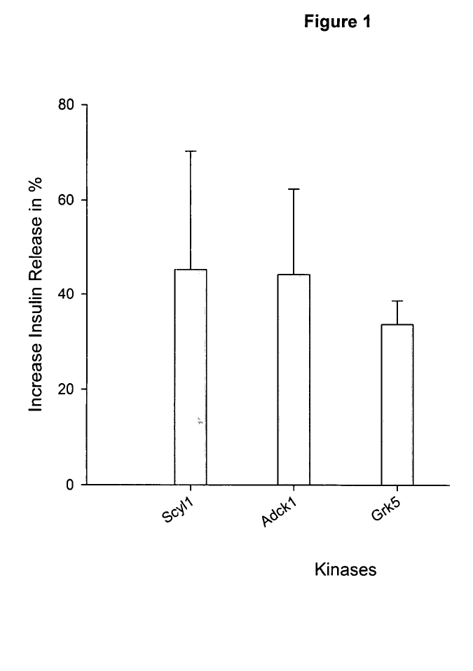

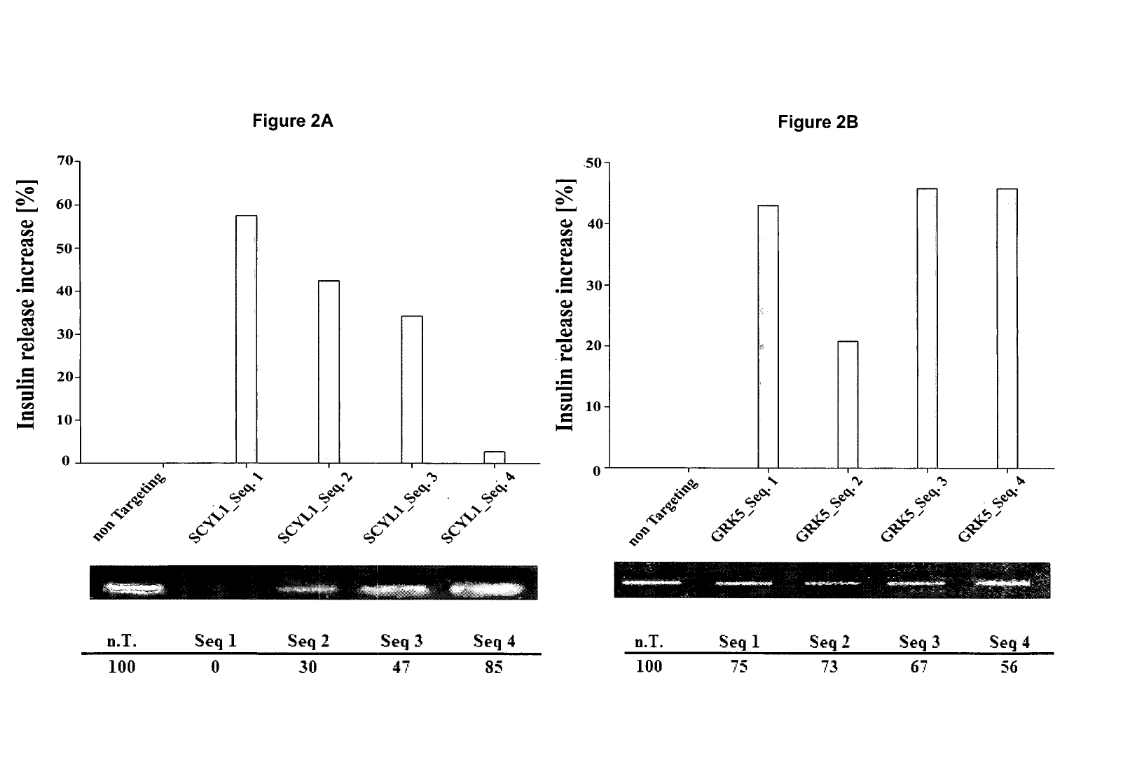

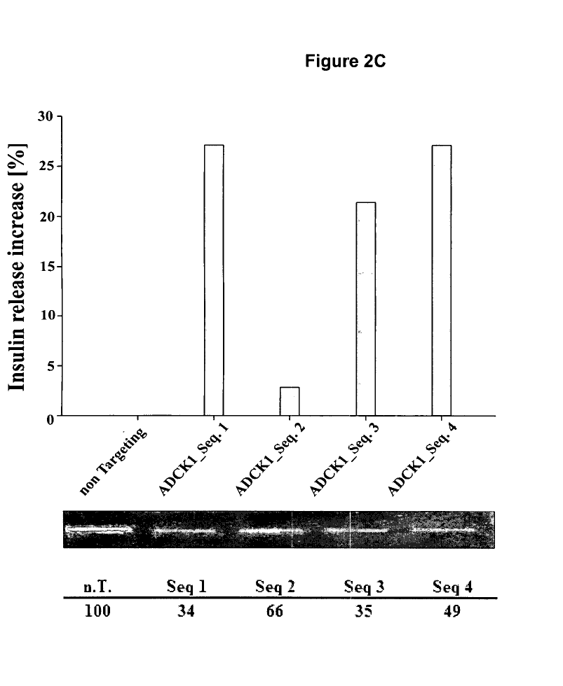

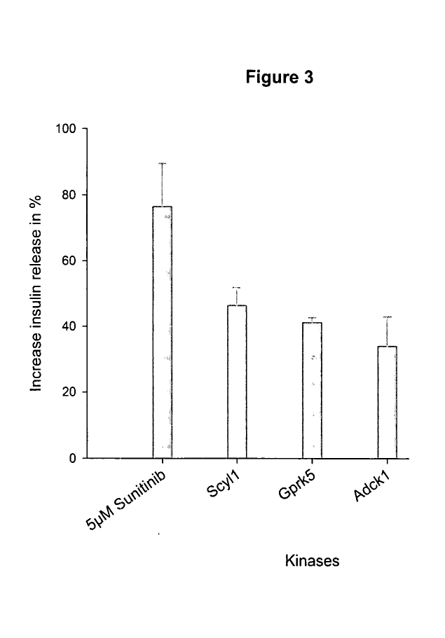

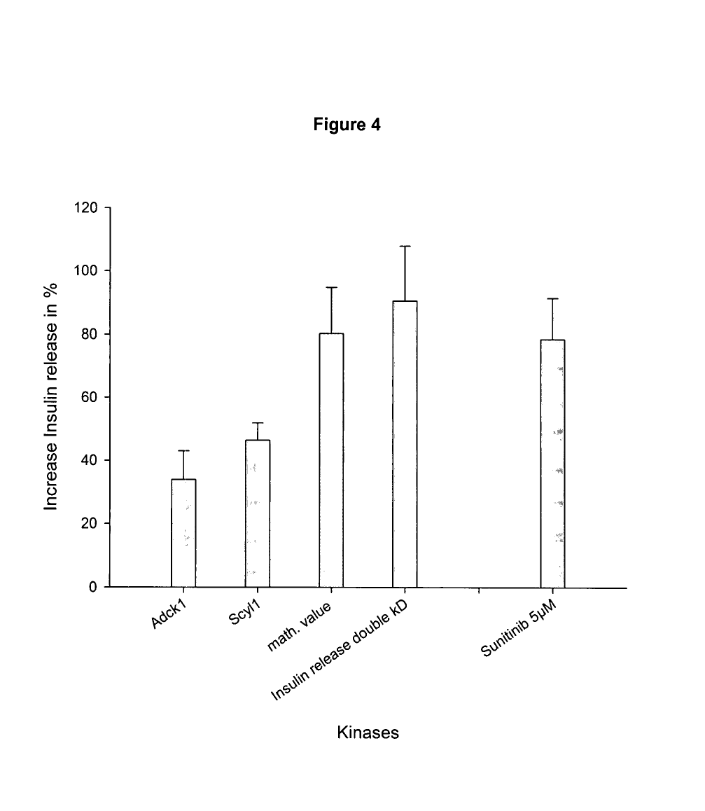

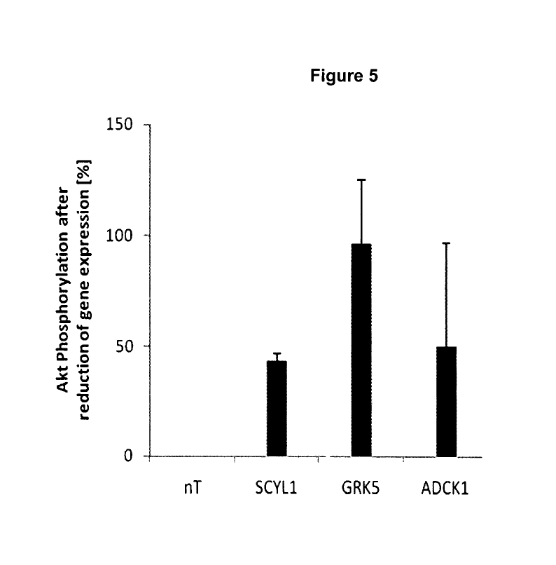

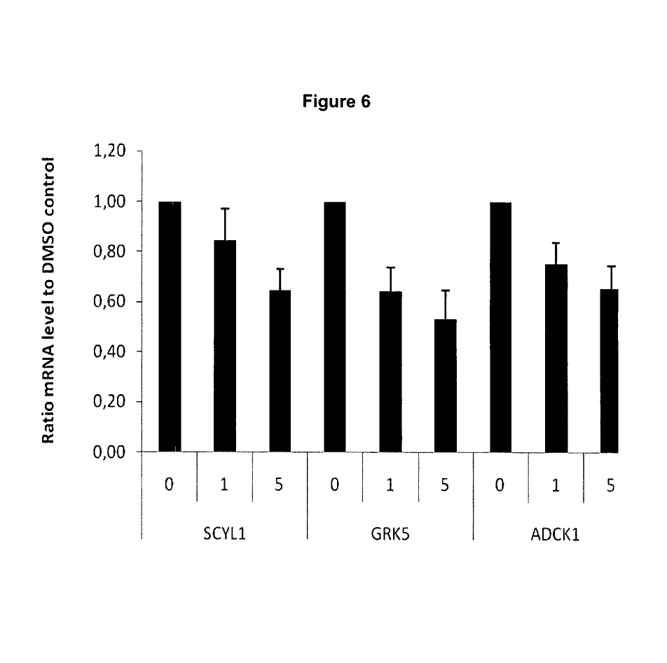

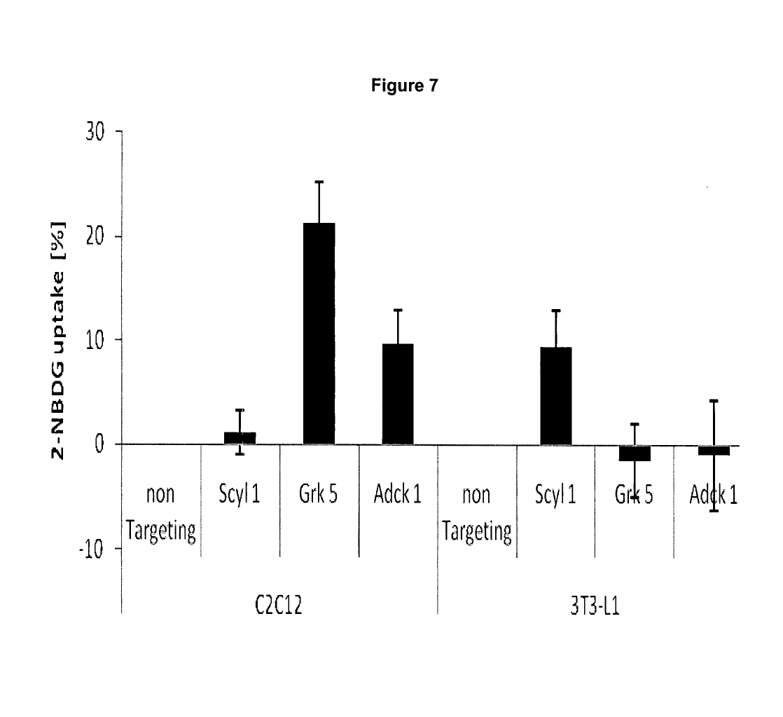

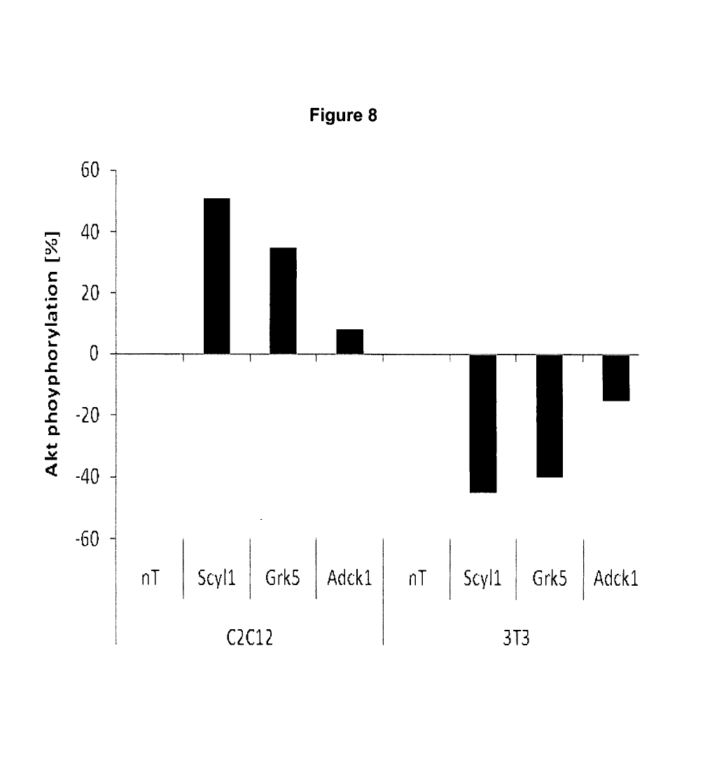

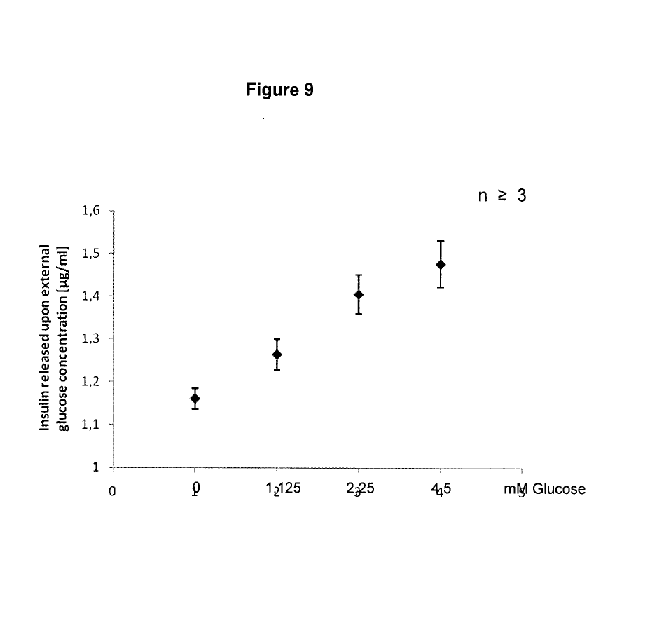

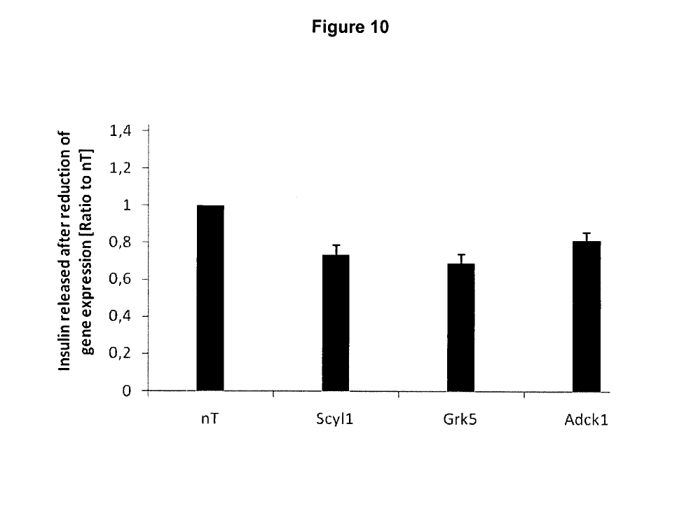

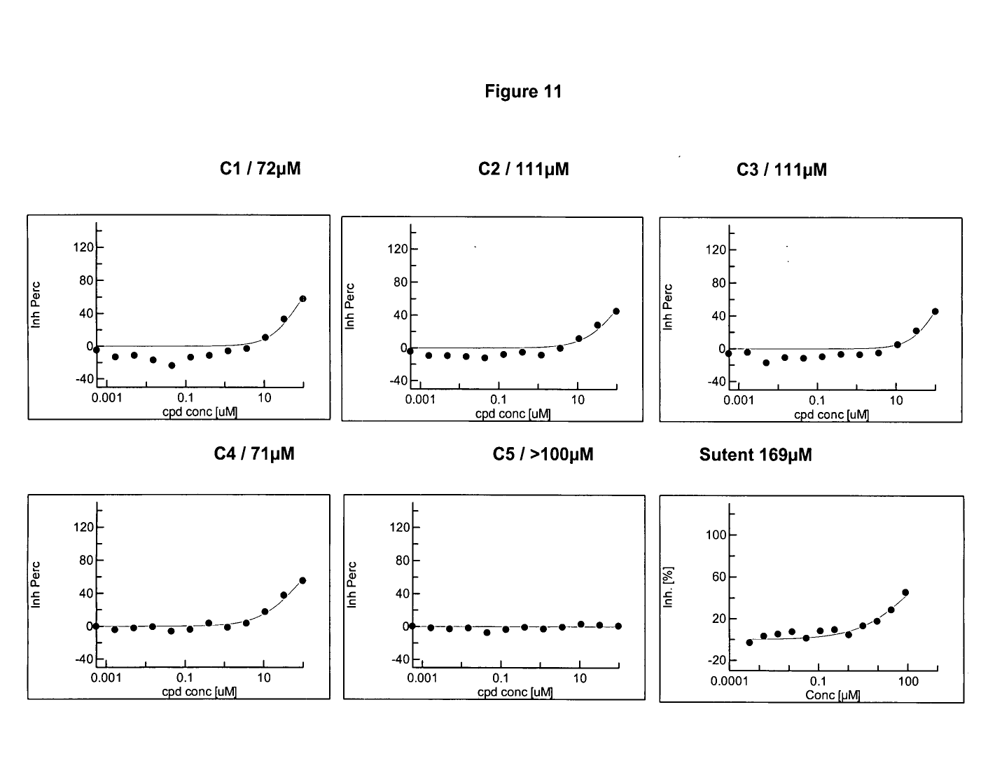

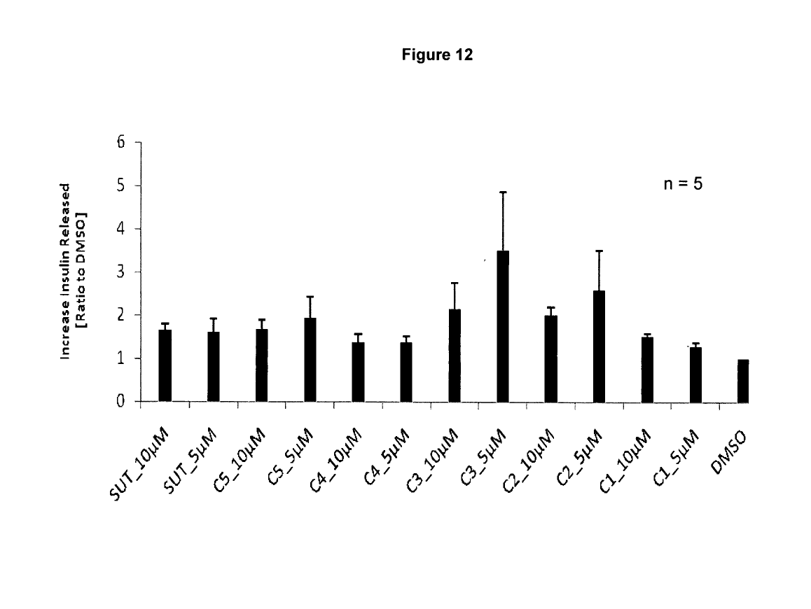

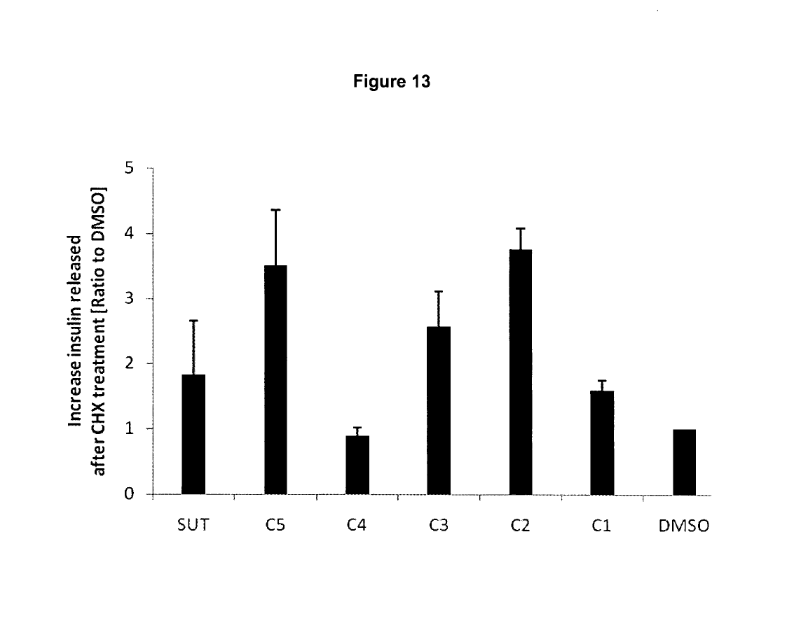

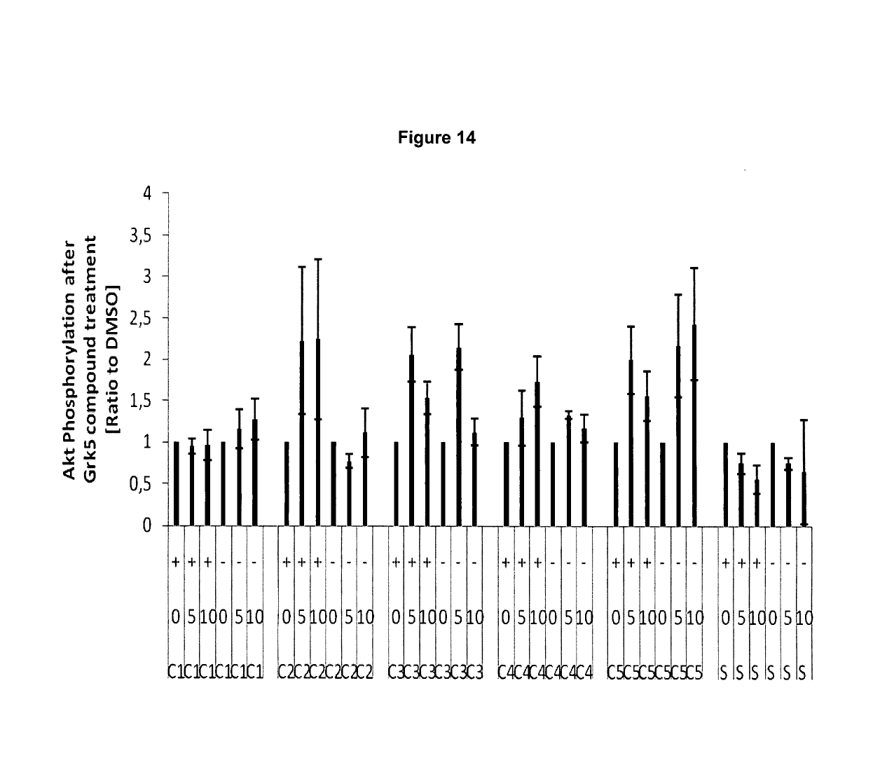

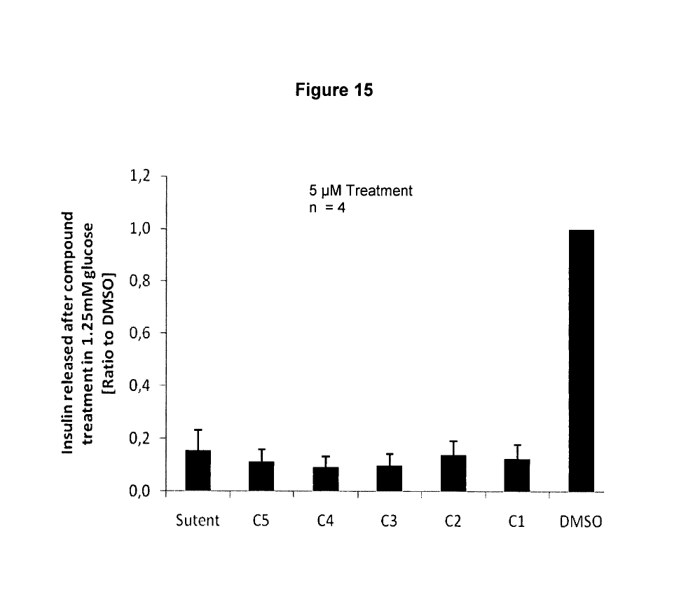

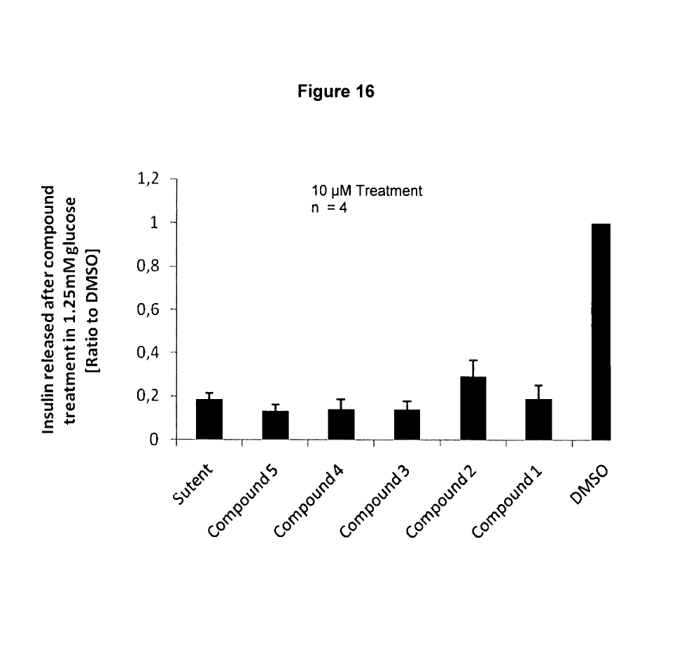

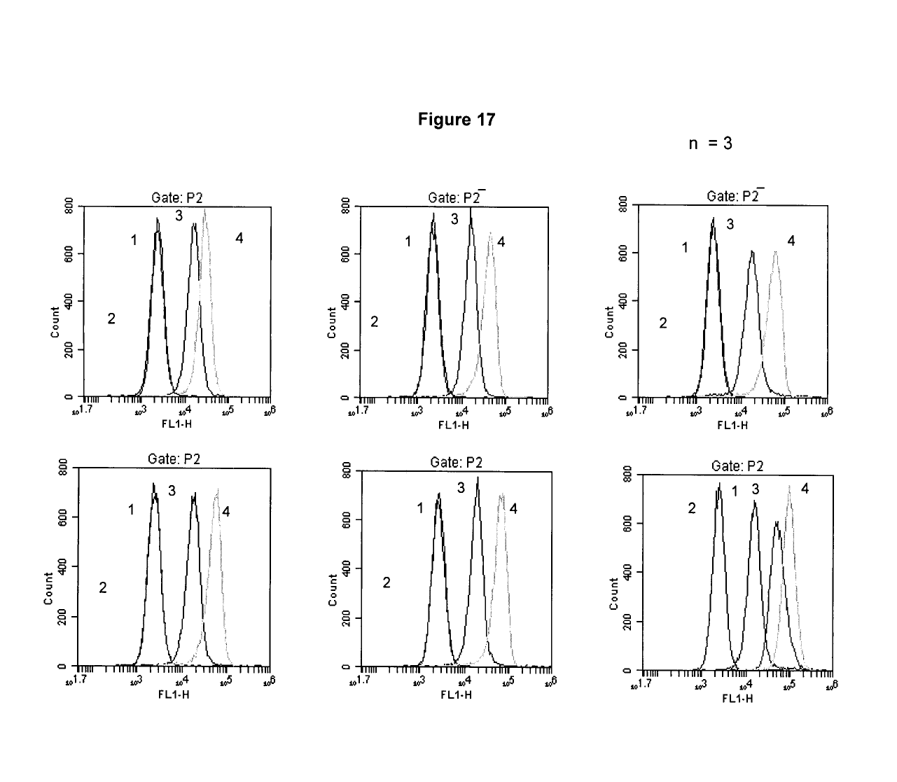

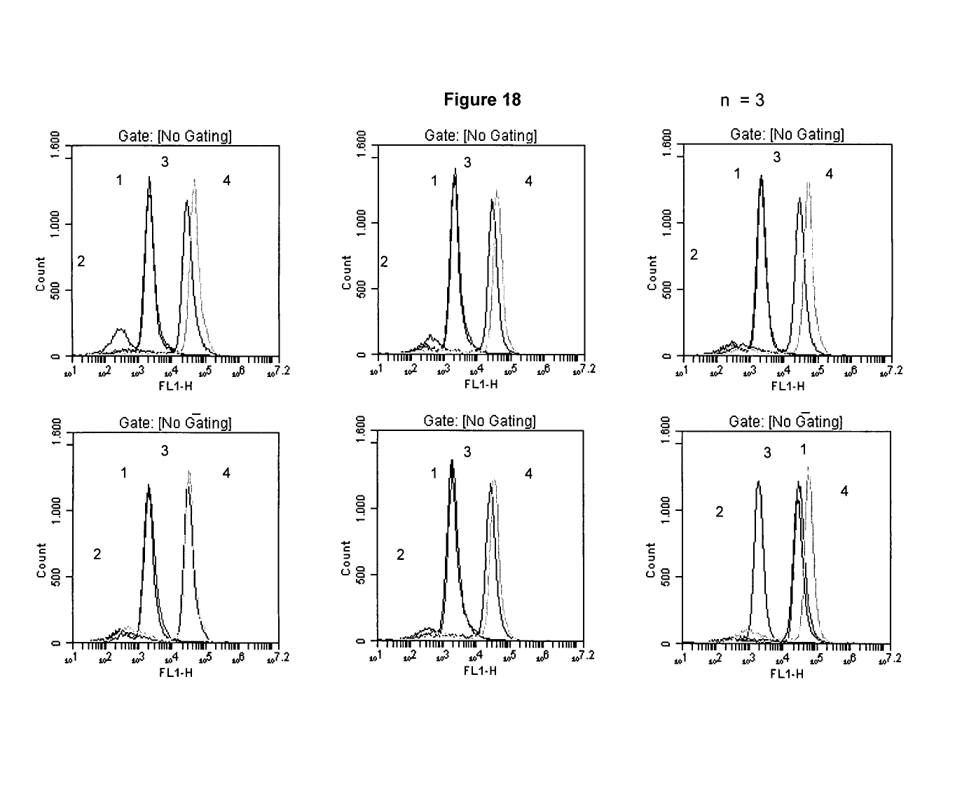

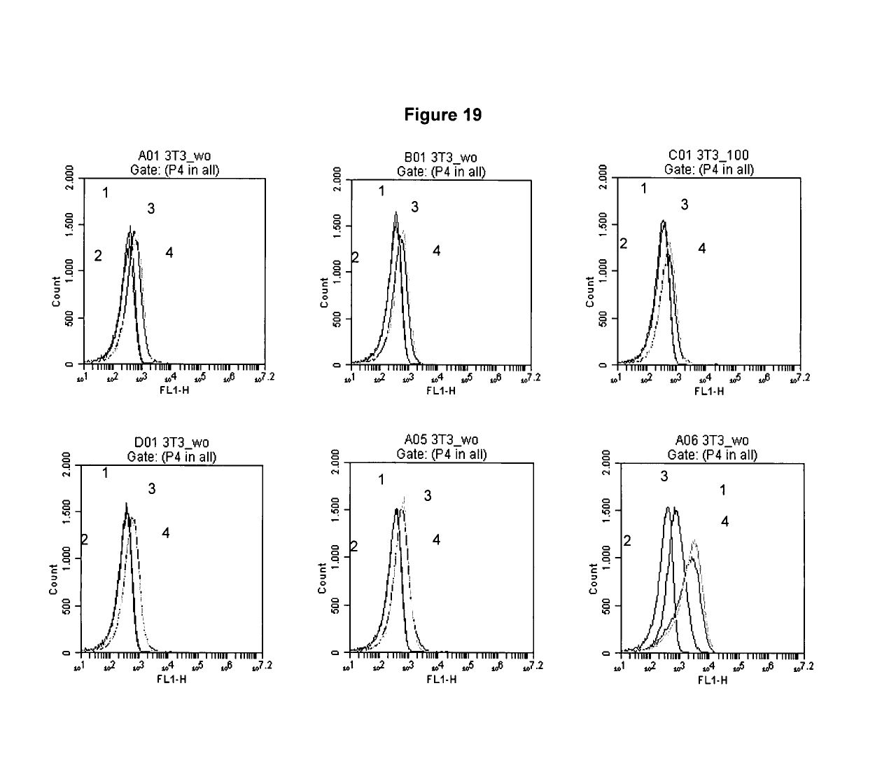

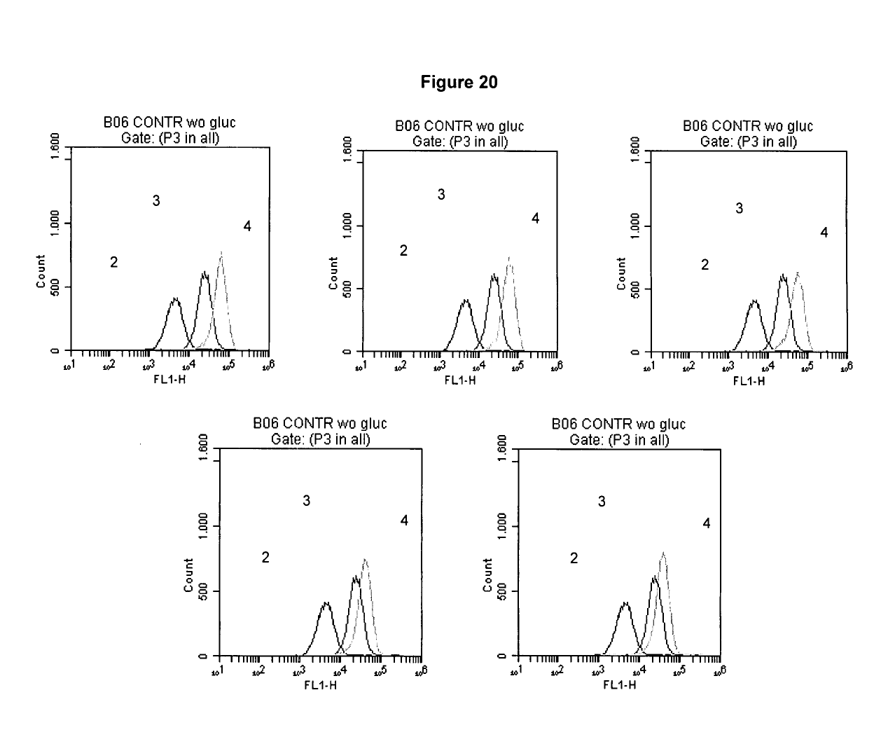

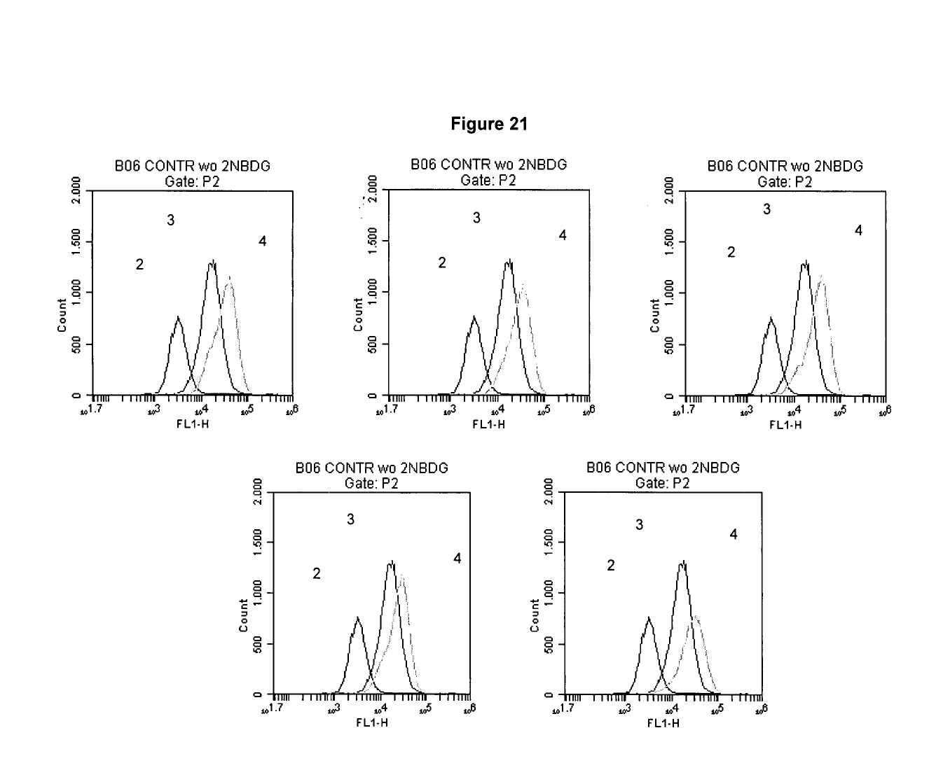

1. A modulator for
a) inhibition of at least one of the protein kinases selected from the group consisting of SCYL1, ADCK1, and GRK5 or
b) inactivation, degradation, downregulation, intercalation of at least one nucleic acid selected from the group consisting of the nucleic acid encoding SCYL1, the nucleic acid encoding ADCK1, and the nucleic acid encoding GRK5,
for the treatment of a disease of the carbohydrate metabolism.

2. The modulator according to claim 1, wherein the disease is diabetes.

3. The modulator according to claim 1, wherein the disease is diabetes mellitus type 2.

4. The modulator according to claim 1, wherein said modulator is used for the up-regulation of insulin production and/or release of insulin.

5. The modulator according to claim 1, wherein the modulator is
a) a small molecule,
b) an RNA molecule,
c) an siRNA molecule, an miRNA molecule, or a precursor thereof,
d) an antisense oligonucleotide,
e) an aptamer
f) a polypeptide,
g) an antibody, or
h) a ribozyme.

6. A pharmaceutical composition comprising the modulator of claim 1 for the treatment of a disease of the carbohydrate metabolism.

7. A pharmaceutical composition according to claim 6, wherein the disease is diabetes.

8. A pharmaceutical composition according to claim 6, wherein the disease is diabetes mellitus type 2.

9. A pharmaceutical composition according to claim 6, used for the up-regulation of insulin production and/or release of insulin.

10. A method for screening for a modulator for treatment of a disease of the carbohydrate metabolism, the method comprising
a) contacting a test compound with at least one polypeptide selected from the group consisting of SCYL1, ADCK1, and GRK5 polypeptide,
b) detecting the binding of said test compound to the SCYL1, ADCK1, or GRK5 polypeptide, and
c) determining the activity of the SCYL1, ADCK1, or GRK5 polypeptide in the presence of said test compound.

11. The method according to claim 10, wherein instead of polypeptides nucleic acids encoding the polypeptides are used and the expression rate is determined instead of the activity.

12. The method according to claim 10, wherein the test compound is RNA or a peptide or an antibody or a kinase inhibitor.

13. A kit comprising
a) SCYL1, ADCK1, and/or GRK5 polypeptides,
b) a nucleic acid encoding SCYL1, a nucleic acid encoding ADCK1, and a nucleic acid encoding GRK5,
c) a cell line with insulin production,
d) a control compound known to affect the insulin production by binding the SCYL1, ADCK1, and/or GRK5 polypeptide or the corresponding nucleic acid.

14. A method for treatment of a metabolic disease comprising:
administering a subject in need thereof a therapeutically effective amount of at least one modulator for:
a) inhibition or activation of at least one of the tyrosine kinases selected from the group consisting of SCYL1, ADCK1, and GRK5, or
b) inactivation, degradation, downregulation, intercalation or activation of at least one nucleic acid selected from the group consisting of the nucleic acid encoding SCYL1, the nucleic acid encoding ADCK1, and the nucleic acid encoding GRK5.


SEQUENCE LISTING (12 sequences)

SEQ ID NO: 1 - DNA, 23 bp, Mus musculus
cggcggcgacgatgtggttcttt

SEQ ID NO: 2 - DNA, 22 bp, Mus musculus
cggcgttgccctgtgccgagta

SEQ ID NO: 3 - DNA, 23 bp, Mus musculus
ctgacacgggcaaggctgagatt

SEQ ID NO: 4 - DNA, 23 bp, Mus musculus
gcgccctgatacaacaccgagac

SEQ ID NO: 5 - DNA, 22 bp, Mus musculus
gccgggtgctggagactgagga

SEQ ID NO: 6 - DNA, 24 bp, Mus musculus
tggcggttctggaggctgacttct

SEQ ID NO: 7 - DNA, 2637 bp, Mus musculus
cccgccccgccccggctcgggcggccggaggacccggagtggaggctcccgagcccgtggcggcggcgacgatgtggttctttgcccgggacccggtccgggacttcccgttcgagctgagccctgagccccccgaaggcgggccgcccgggccctggatcctgcaccgaggccgcaaaaaggccacaggcagcgcagtgtccatcttcgtgtatgatgtgaaaccgggagctgaagagcagacccaggtggccaaagctgccttcaaacgcctcaaaactctccgacaccccaacatcctggcctatatcgatgggttggagacagaaaagtgcctccacatcgtgacagaggctgtgacccccctgggaacatacctcaaggcacgagcagaagcaggtggcctgaaggagcaggagctgtcatgggggttacaccagatcgtgaaagccctcagcttcctggtcaacgactgcaacctcatccacaataatgtctgcatggccgctgtgtttgtggacagggctggcgagtggaaacttgggggtctggactacatgtactcggcacagggcaacggcgggggaccacccagcaaggggatcccggagctcgagcagtatgatcccccggagctggctgacagcagtagcagagcagtcagagagaagtggtcagcagacatgtggcgcttgggctgcctcatctgggaagttttcaatgggtctctacctcgggcagctgccctgcgcaaccctgggaagatccccaaatccctggtgacccattactgtgaactggtgggagctaacccaaaagtacgtcccaacccggcccgcttcctgcagaactgccgggcacccggtggcttcatgagcaaccgctttgttgagaccaacctcttcctggaggagattcagatcaaagagccagctgagaagcagaagttcttccaagagctgagcaagagtctagactcatttcccgaagatttctgtcgacacaaggtgctgccccagctactgactgcctttgagtttggcaatgctggggccgtggtcctcacacctctcttcaaggtgggaaaatccctccgtgctgaagagtaccaggagaagatcatccccgtggtagttaagatgttctcatccaccgaccgggccatgcgcatccgcctcctccagcagatggagcagttcatccaataccttgatgagccaacagtcaacacgcagattttcccccacgtcacacatggcttcctggacaccaaccccgccatccgcgagcagacggtcaagtccatgctgctcttggccccaaagctgaatgaggccaatctcaatgtggaactgatgaagcactttgcaaggctacaagccaaggacgaccagggtcctatccgctgcaacaccacggtctgcttgggcaaaatcggctcctatctcagtgctagtactagacacagggtcctcacctccgccttcagcagagccactaaggacccatttgcaccatcccgggttgcgggtgtcctgggctttgctgccacacacaatctctattcgatggacgactgtgcccataagatcctgcctgtgctctgtggccttactgtggaccctgagaaatctgtgcgggaccaggcctttaagaccattcgaagcttcctgtccaaattagagtctgtgtcagaggatcccacccagctggcagaagtagagaaggatgtccatgcagcgtccagtcctggaacaggaggagctgcagccagctgggcaggctgggctgtgactggggtatcctctctcacctccaagctgatccgagcacaccccacgcctgtgccgtctgataccactgtgccccagagaccagtgccagagggaaatcctgctccagcccctgcccttgcccaagctatccctgcaacctcagggcactgggagacacaggaagacaaggacactgcagaagacagcgccactgctgacagatgggacgatgaggactggggcagcttggagcaggaagctgaatccgtgttggcacagcaggatgactggagtgccaagggccaaggaagccgagctggacagatcaaccacccagaccacaaatctctggaatcacattggagcagctgggaagttgagggctcctgggaccagggctggcaggaacccagctctgtggagccacctccagaaggcactcggctagctagcgaatataactggggtggtgcagagcccagtgacaagggcgacccctttgctgccctgtctgttcgtcccagcgctcagcccaggccagacccagactcctggggtgaagacaactgggaaggcttggaggctgagagcagacaggtaaaggcagagctggcccggaaaaagcgagaggaaaggagaagagaaatggaagccaaacgggcagagaaaaagaccaccaaggggcccatgaagctgggagcccggaagctggactgacaaccccacccccaagccactgggcttccaaccactggagagcaggcccggcggatgtatttattgtacaaaccatgtgagcctggtcagcaggtcaggcacatctagtgtacataatcagagccacaataaattctatttcaca

SEQ ID NO: 8 - DNA, 3182 bp, Mus musculus
gcggccgccaggtcgccccgggctggccgaaccccttgctgcccggaaacaaagtgcacctttgtcagagacctggcgcgtgtgagcggcagacggcgggccgcgcggaaccccaggagcgcgccttcgctcgggtctcagctcactcctgggcctcccacagcagcgttctgccgccttggcttgtgctttccgctttctcggatcttggcgggacaggaaagggactctgcgctccaggagtgggggtttcgttttggctgagtcgtttttgactcctgccggcgaggctgggccgcctgggctcagcgccataccggcagcagtcctgctccacagtcccggtgcaatagagccctcgccccgtcagacgcgggactacaattcccagcactcctcgcgttcagagcgcgcggtgggagttgcctccgggcagacgcctgcgcgctgcagcggctcgagcctggagtaccacacccccgggaggggaacgaggggaggctgaagcatccgaagcattaaagcatccgagggagccggaggggaggagaatggagtgacagagacgcgcggagggtggggggtgggggggaaagtgttgagggaggggggaggggggacacagagggaggaagaagcggcggcggcgtctcttcggtgcagagggggaaactccgcgggctccgagaaagaataatgcggtagcaggcaggctgcttgctctggggtttggcagcagcggcggcagctcgagcagtggcagcggcagcggcatcaccccagacgctgacagcacagccggccggctccctcgctgactgccgactgtcaatggagctggaaaacatcgtggccaacacggtcttgctgaaagcccgggaagggggtggaggaaagcgcaaagggaaaagcaagaagtggaaggaaatcctgaagtttcctcacatcagccagtgtgaagacctccgaaggaccatagacagagattactacagtctatgtgacaagcaaccaattgggagactgctttttcgacagttctgtgaaaccaggcctgggctggagtgctacattcagttcctggacttagtggcagaatatgaaattactccagatgaaaaccttggggcgaaggggaaggaaataatgaccaagtacctcactccaaagtccccagtcttcattgcccaagttggacaggacctggtctcccagacagagaagaagctcctgcagagcccctgcaaagaactcttctctgcttgtgctcagtctgtccatgactacttgaagggagaccccttccacgagtacctggatagcatgtattttgaccgttttctgcagtggaaatggttagaaagacaaccagtgaccaaaaacactttccggcagtaccgagtgctgggcaaagggggctttggagaggtctgtgcctgccaggttcgggccactggtaaaatgtatgcttgtaaacgcttagagaagaagaggatcaaaaagaggaaaggcgaatccatggcactcaacgaaaagcagattcttgagaaggtcaacagccagtttgtggtcaacctggcctatgcctatgaaaccaaagatgcactatgcctggttctgaccattatgaatggtggtgacctgaagtttcacatctacaatatggggaatcctggctttgaggaagagcgagccttattttatgcagctgagatcctctgtggcctagaagacttacaccgtgagaacactgtctatagagatctaaaacccgaaaacatcttgctggatgattatggccacataaggatctcagacctcggactggccgtgaagatccccgagggagaccttatccgtggccgggtaggcactgttggctacatggccccagaagttctgaacaaccagcgatatggactgagccctgactactggggcctgggctgcctcatctatgagatgattgaaggccagtcaccatttcgaggtcgcaaggagaaggttaagcgggaagaggtggatcgccgggtgctggagactgaggaagtgtattcctccaagttctctgaagaggccaagtccatctgcaacatgctgctcaccaaagactcgaagcagaggctgggctgccaggaggagggggccgccgaggtcaagaggcaccctttcttcaggaacatgaactttaagcgcctggaggctgggatgttggaccctcccttcgttccagatccccgggctgtatactgcaaggatgtgctggacattgagcagttctccactgtgaaaggtgtcaacctggaccatacggacgatgatttttactcaaagttctctacaggctctgtgccaattccatggcaaaatgagatgatagaaacagaatgtttcaaggagctgaatgtgttcggacctaacggtaccctctcaccagacctgaacagaagtcagcctccagaaccgccaaagaaagggctgttccacagactcttcaggcgtcagcatcaaagcaattccaagagttcacctactcctaagaccagttgtaaccaccgaataaattcaaaccacatcaattcaaactccactggaagcagctagtttcggctctggccttgaagtcaaaagtggaaccagctcagagccttctacttggaagcagaacttgtagccaggggagcttccactgtggctcagtggccagcaaagctccagtgggaactaagataggagaccgttcccccaataacaaacctccaagtttctcaaagaaatttccactcaggtctgttttccaaggtggccccaagctggggtggactagaattttccttgttgaacattgcaatagaaacccaatgggatatgacagcttgcacggattttaatagcatcctaactagaactgaattttgtctttattatttttaaaggaaagttttgtaaatttctctattgtctctgtttacattttgtatatttgtatttaagtgaaagtcagactttgagggtgtatattttctgtgcagccactgttaagccatgtgttttaagacattttagattggagggggggttacaaaaatgtgactctagacttccagagcctcaaaagagataatgtttttattaaatatagaaaatatctcactttttacctttaaaaaaaaaaaaaaaaa

SEQ ID NO: 9 - DNA, 2287 bp, Mus musculus
gcccccttggctgtgactctctcgctcggtgtcctagaggtccttagtgtggactggccacttgggtggcccaaccttgcgtgccctggagtaggcagaggtgaagaggggctacgagtggctccgccggcagccttcagacatcggtgaccttgtgggctcccacgcagagggtctggagacatggccagaaaggctctcaagcttgcttcatggaccagcgtggctcttgctgcctccggtgtctacctctacagtaacaactacttggaccctaatgactttggcgctgtcagggtgggcagagctgttgctacgacagctgtcatcagctatgactacctcacctccctgaggagtgtcccatatggctctgaggagtatttgcagcgtcgatcccaggtgcacctccgctctgccaggcgtctctttgagctctgctgtgccaaccggggcactttcatcaaggtgggccagcacctgggggcgctggactacctgctgccagaagagtacaccagcacactgaaggtgttgcacagtcaagccccacagagcagcatgcaagaggtccggcaggtcatccgagaagacctgggcaaggagatccacgatttgttcctgagcttcgatgacacccctcttggggcagcctccctggcccaggtccacaaggcggtgttgcatgatggtcggacagtggcagttaaggtccagcacccgaaggttcaggctcaaagctctaaggacattctcctgatggaggtgcttgtcctggccgtgaagcaacttttcccagattttgaattcatgtggctggtggatgaagcgaagaagaacctgcctctcgagttggacttcctgaatgaagggaggaacgctgagaaagtggcccacatgctcaggcactttgacttcctaaaggttccccagatccactgggagctgtctaccaagagggtgctcctgatggaatttgtagagggaggtcaagtcaacgacagggcctacatggagaagaaccagatcgatgtgaatgagatctcctgccacctgggcaagatgtacagtgagatgatctttgtcaatggcttcgtgcactgtgacccccacccaggcaacgtactggtacggaagcgtcctgacacgggcaaggctgagattgtcctcttagaccatgggctttaccaggtgctcacggaggagttccgcctggactactgccatctgtggcagtctctgatctggactgacatggacgggctgaaacagtacagccagcgcctgggagctgcagacctctacccactgtttgcctgtatgctgacagcccggtcctgggactcagtcaaacagggcattgggcaagctccagtctctgctactgaggactcagagattcgcaataatgcagcctgctacctgcctgagatcagccagctccttaaccatgtgcctcgccagatgctgctcatcctgaagactaatgatctactccgtagcattgagaccaccctgggcacgcgctccagtgccagttccttcctcaacatgtctcggtgttgtatcagggcgctggctgaacacaagaagagggatgccggctctttcttcagaaggactcagatatctttcagtgaggcctttagcctgtggcagatcaaccttcatgaacttctgcttcgagtgagggccttgaggctagcttgctgggtctcagctctcctgggctggctgactcgggctccacacagaatgtgatggtctttccctgccccttcgtagtgtctttccacacctcattccttccttcacgctgggacgacccactgacccatggctgcctagggttggctgtggtcccagagtggtcatccatggcaccctcatgctctgccattggagcccctgccttggggtggccttgtcttgaggtcctggaatgtcctggagagtgagatgggataaagcaacctctctcccatctcctgtgtgtgccattgacttggtcatccctacttttatgaggactgtgagaatggaccaaccacctgtgtcacaggggcgtgtttaacttgtagggaataagtagaaactcagaacctgcagagaacagactttatatttttgctgtatcactcccaaagttgtctcgcctcagtgaggaagactgcatctgagagggaacgatgcaactgtgggctcatgctgtcatggtgacaccttcagtgttatatgtattgtatatattgtttattgtaataaaccaataaacagtttcaaggtt

SEQ ID NO: 10 - protein, 806 aa, Mus musculus
MWFFARDPVRDFPFELSPEPPEGGPPGPWILHRGRKKATGSAVSIFVYDVKPGAEEQTQVAKAAFKRLKTLRHPNILAYIDGLETEKCLHIVTEAVTPLGTYLKARAEAGGLKEQELSWGLHQIVKALSFLVNDCNLIHNNVCMAAVFVDRAGEWKLGGLDYMYSAQGNGGGPPSKGIPELEQYDPPELADSSSRAVREKWSADMWRLGCLIWEVFNGSLPRAAALRNPGKIPKSLVTHYCELVGANPKVRPNPARFLQNCRAPGGFMSNRFVETNLFLEEIQIKEPAEKQKFFQELSKSLDSFPEDFCRHKVLPQLLTAFEFGNAGAVVLTPLFKVGKSLRAEEYQEKIIPVVVKMFSSTDRAMRIRLLQQMEQFIQYLDEPTVNTQIFPHVTHGFLDTNPAIREQTVKSMLLLAPKLNEANLNVELMKHFARLQAKDDQGPIRCNTTVCLGKIGSYLSASTRHRVLTSAFSRATKDPFAPSRVAGVLGFAATHNLYSMDDCAHKILPVLCGLTVDPEKSVRDQAFKTIRSFLSKLESVSEDPTQLAEVEKDVHAASSPGTGGAAASWAGWAVTGVSSLTSKLIRAHPTPVPSDTTVPQRPVPEGNPAPAPALAQAIPATSGHWETQEDKDTAEDSATADRWDDEDWGSLEQEAESVLAQQDDWSAKGQGSRAGQINHPDHKSLESHWSSWEVEGSWDQGWQEPSSVEPPPEGTRLASEYNWGGAEPSDKGDPFAALSVRPSAQPRPDPDSWGEDNWEGLEAESRQVKAELARKKREERRREMEAKRAEKKTTKGPMKLGARKLD

SEQ ID NO: 11 - protein, 590 aa, Mus musculus
MELENIVANTVLLKAREGGGGKRKGKSKKWKEILKFPHISQCEDLRRTIDRDYYSLCDKQPIGRLLFRQFCETRPGLECYIQFLDLVAEYEITPDENLGAKGKEIMTKYLTPKSPVFIAQVGQDLVSQTEKKLLQSPCKELFSACAQSVHDYLKGDPFHEYLDSMYFDRFLQWKWLERQPVTKNTFRQYRVLGKGGFGEVCACQVRATGKMYACKRLEKKRIKKRKGESMALNEKQILEKVNSQFVVNLAYAYETKDALCLVLTIMNGGDLKFHIYNMGNPGFEEERALFYAAEILCGLEDLHRENTVYRDLKPENILLDDYGHIRISDLGLAVKIPEGDLIRGRVGTVGYMAPEVLNNQRYGLSPDYWGLGCLIYEMIEGQSPFRGRKEKVKREEVDRRVLETEEVYSSKFSEEAKSICNMLLTKDSKQRLGCQEEGAAEVKRHPFFRNMNFKRLEAGMLDPPFVPDPRAVYCKDVLDIEQFSTVKGVNLDHTDDDFYSKFSTGSVPIPWQNEMIETECFKELNVFGPNGTLSPDLNRSQPPEPPKKGLFHRLFRRQHQSNSKSSPTPKTSCNHRINSNHINSNSTGSS

SEQ ID NO: 12 - protein, 551 aa, Mus musculus
MARKALKLASWTSVALAASGVYLYSNNYLDPNDFGAVRVGRAVATTAVISYDYLTSLRSVPYGSEEYLQRRSQVHLRSARRLFELCCANRGTFIKVGQHLGALDYLLPEEYTSTLKVLHSQAPQSSMQEVRQVIREDLGKEIHDLFLSFDDTPLGAASLAQVHKAVLHDGRTVAVKVQHPKVQAQSSKDILLMEVLVLAVKQLFPDFEFMWLVDEAKKNLPLELDFLNEGRNAEKVAHMLRHFDFLKVPQIHWELSTKRVLLMEFVEGGQVNDRAYMEKNQIDVNEISCHLGKMYSEMIFVNGFVHCDPHPGNVLVRKRPDTGKAEIVLLDHGLYQVLTEEFRLDYCHLWQSLIWTDMDGLKQYSQRLGAADLYPLFACMLTARSWDSVKQGIGQAPVSATEDSEIRNNAACYLPEISQLLNHVPRQMLLILKTNDLLRSIETTLGTRSSASSFLNMSRCCIRALAEHKKRDAGSFFRRTQISFSEAFSLWQINLHELLLRVRALRLACWVSALLGWLTRAPHRMRREMEAKRAEKKTTKGPMKLGARKLD